(19) United States
(12) Patent Application Publication
(10) Pub. No.: US 2024/0197853 A1
(43) Pub. Date: Jun. 20, 2024

(54) MULTIVALENT CHOLERA MULTIEPITOPE FUSION ANTIGEN (MEFA) AND METHODS OF USE

(71) Applicant: The Board of Trustees of the University of Illinois, Urbana, IL (US)

(72) Inventor: Weiping Zhang, Savoy, IL (US)

(73) Assignee: The Board of Trustees of the University of Illinois, Urbana, IL (US)

(21) Appl. No.: 18/554,423

(22) PCT Filed: Apr. 5, 2022
(86) PCT No.: PCT/US2022/023521

§ 371 Date: Oct. 6, 2023

Related U.S. Application Data
(60) Provisional application No. 63/171,410, filed on Apr. 6, 2021.

(51) Int. Cl. A61K 39/02; A61K 39/00; A61P 31/04; A61P 37/04; C07K 14/28

(52) U.S. Cl. CPC A61K 39/107; A61P 31/04; A61P 37/04; C07K 14/28; A61K 2039/522

(57) ABSTRACT

Compositions and methods for eliciting an immune response in a subject against Vibrio cholerae are provided. The compositions include fusion proteins with one or more epitopes from Vibrio cholerae, nucleic acids and vectors encoding the fusion proteins, pharmaceutical compositions, immunogenic compositions, and vaccines. In examples, the methods include administering a disclosed composition to a subject, such as a human.

### CROSS-REFERENCE TO RELATED APPLICATIONS

This claims the benefit of U.S. Provisional Application No. 63/171,410, filed Apr. 6, 2021, herein incorporated by reference in its entirety.

### FIELD

This application provides Vibrio cholerae multi-epitope fusion proteins and compositions and methods of use, particularly for inducing an immune response against Vibrio cholerae in a subject.

### BACKGROUND

Vibrio cholerae infection remains a public health threat, especially in South and Southeast Asia and sub-Sahara Africa (World Health Organization, who.int/news-room/fact-sheets/detail/cholera; visited Mar. 25, 2021). V. cholerae infection causes 1.3 to 4 million clinical cholera cases and 21,000 to 143,000 deaths globally each year. About half of the cases and deaths are children aged less than five years (Ali et al. (2015) PLoS Neglected Tropical Diseases, 9(6): e000383). Cholera cases and deaths are also associated with poverty among the poorest individuals (World Health Organization (2017) Ending Cholera—A Global Roadmap to 2030, who.int/cholera/publications/global-roadmap/en/; visited Mar. 25, 2021). In Bangladesh, V. cholerae is one of the top three leading etiologies of diarrhea requiring hospitalization across all ages and geographies (Taniuchi et al. (2020) Clin Infect Dis., doi:10.1093/cid/ciaa840).

Heterogeneity of V. cholerae strains, with over 200 currently identified serogroups, is a challenge for cholera prevention. Since O1 (classical biotype for the first six pandemics and biotype El Tor for the seventh pandemic) and O139 (massive cholera-like outbreaks in early 1990s then thought of as the 8^{th} pandemic, but waned) have been associated with epidemic and pandemic cholera and are responsible for the vast majority of clinical cases (Kaper et al. (1995) Clinical Microbiology Reviews, 8:48-86; Sack et al. (2004) Lancet, 363:223-233). Thus, O1 and O139 serogroups are the main targets in cholera vaccine development. However, non-O1 and non-O139 (non-O1/non-O139) serogroup strains also cause severe gastrointestinal infections sporadically (Deshayes et al. (2015) Springerplus, 4:575), thus an ideal cholera vaccine protects against multiple major serogroups that cause outbreaks.

Currently, three whole-cell oral cholera vaccines (OCVs) are prequalified by World Health Organization and are licensed to many countries (Baker-Austin et al. (2018) Nature Reviews Disease Primers, 4:1-19; World Health Organization. 2020. Prequalified Vaccines. extranet.who.int/gavi/PQ_WEB. Accessed). While OCVs confer >60% protective efficacy in adults and children over five years of age (Longini et al. (2007) PLoS Medicine, 4:1776-1783; Bhattacharya et al. (2013) Lancet Infectious Diseases, 13:1011-1011; Seo et al. (2020) Gut Microbes, 11:1486-1517), they provide significantly less or minimal protection to children under five years of age, especially those living in endemic regions (Clemens, et al. (1990) Lancet, 335:270-3; Bi et al. (2017) Lancet Infectious Diseases, 17:1080-1088; Luquero and Azman (2018) Lancet Infectious Diseases, 18:947-948; Qadri et al. (2018) Lancet Infectious Diseases, 18:666-674), thus improved vaccines are greatly needed.

### SUMMARY

Heterogeneity of Vibrio cholerae strains is a challenge for cholera prevention. A vaccine targeting a broad range of virulence determinants may provide cross protection against multiple Vibrio cholerae serogroups. Described herein are multivalent cholera MEFA (multiepitope fusion antigen) proteins and their use to confer broad immunogenicity and cross-protective antibodies against cholera.

Provided herein are fusion proteins including a backbone protein with a consensus sequence having at least 90% sequence identity to SEQ ID NO: 4. In some embodiments, the fusion protein contains at least one heterologous Vibrio cholerae epitope (e.g., heterologous to the backbone protein). In some examples, the at least one heterologous Vibrio cholerae epitope is 8-15 amino acids in length. In other examples, the at least one heterologous Vibrio cholerae epitope is about 9, 10, 11, 12, or 14 amino acids in length. In some examples, the at least one heterologous Vibrio cholerae epitope is a peptide of a Vibrio cholerae virulence factor, for example, toxin coregulated pilus A (TcpA), cholera toxin (CT), cholera toxin subunit B (CTB), LPS O-antigen mimic (LPS), sialidase (Neu; also called NanH), hemolysin A (HlyA), flagellin C (FlaC), or flagellin D (FlaD). In some examples, the at least one heterologous epitope includes one or more of SEQ ID NOs: 6, 8, 10, 14, 16, 18, 20, 22, 24, 31, and 32. In further embodiments, the fusion protein includes at least one homologous Vibrio cholerae epitope (e.g., homologous to the backbone protein), such as a flagellin B subunit (FlaB) epitope. In some examples, the at least one homologous Vibrio cholerae epitope comprises SEQ ID NO: 12. The heterologous and/or homologous epitopes may be contiguous or non-contiguous with one another.

Also disclosed herein are fusion proteins including a backbone protein with a consensus sequence having at least 90% sequence identity to SEQ ID NO: 4, and the fusion protein further comprises at least two Vibrio cholerae epitopes (homologous or heterologous to the backbone protein), wherein at least one of the epitopes is from a different Vibrio cholerae serogroup or biotype than the other epitope. In some examples, the fusion protein contains at least three Vibrio cholerae epitopes, wherein at least two of the epitopes are from a different Vibrio cholerae serogroup or biotype than the third epitope. In some examples, the Vibrio cholerae epitope is a peptide of a Vibrio cholerae virulence factor, for example, toxin coregulated pilus A (TcpA), cholera toxin (CT), cholera toxin subunit B (CTB), LPS O-antigen mimic (LPS), sialidase (Neu; also called NanH), hemolysin A (HlyA), flagellin B (FlaB), flagellin C (FlaC), or flagellin D (FlaD). In some embodiments, the fusion protein includes each of SEQ ID NOs: 6, 8, 10, 12, 14, 16, 18, 20, 22, and 24; or includes each of SEQ ID NOs: 6, 8, 10, 12, 14, 18, 20, 22, 31 and 32. The heterologous and homologous epitopes may be contiguous or non-contiguous with one another.

In some embodiments, the disclosed fusion protein includes a sequence having at least 90% sequence identity to SEQ ID NO: 2, or the fusion protein includes or consists of SEQ ID NO: 2.

Also provided are nucleic acids encoding the disclosed fusion proteins. In some examples, the nucleic acid encodes an amino acid consensus sequence having at least 90% sequence identity to SEQ ID NO: 4, or encodes an amino acid consensus sequence including or consisting of SEQ ID NO: 4. In some embodiments, the nucleic acid encodes at least one heterologous Vibrio cholerae epitope. In some examples, the heterologous Vibrio cholerae epitope is a peptide of a Vibrio cholerae virulence factor (e.g., TcpA, CTA, CTB, LPS, Neu/NanH, HlyA, FlaC, FlaD). In some examples, the nucleic acid also encodes at least one homologous Vibrio cholerae epitope, such as an epitope of FlaB. In some examples, the nucleic acid encodes one or more of SEQ ID NOs: 6, 8, 10, 12, 14, 16, 18, 20, 22, 24, 31 and 32. In some examples, the nucleic acid encodes an amino acid sequence having at least 90% sequence identity to SEQ ID NO: 2, or the nucleic acid encodes an amino acid sequence including or consisting of SEQ ID NO: 2.

Also disclosed are vectors encoding the disclosed nucleic acids. In some examples, the vectors further include at least one nucleic acid encoding at least one peptide of Escherichia coli or Shigella, such as in an additional fusion protein, for example, a fusion protein including enterotoxigenic E. coli antigens CFA/I/II/IV MEFA (e.g., SEQ ID NO: 26), toxoid fusion 3xSTa_{N12S}-mnLT_{R92G/L211A} (e.g., SEQ ID NO: 28), or Shigella MEFA (e.g. SEQ ID NO: 30). Also disclosed are host cells transformed with the vectors including the disclosed nucleic acids.

Disclosed herein are pharmaceutical compositions, which can include a pharmaceutically acceptable carrier and any of the fusion proteins, or nucleic acids or vectors encoding the fusion proteins, as disclosed herein. In some examples, the pharmaceutical compositions include an adjuvant. Also disclosed are immunogenic compositions that include any of the fusion proteins, nucleic acids or vectors encoding the fusion protein, or pharmaceutical compositions disclosed herein. In some examples, the immunogenic composition further includes an additional composition for inducing an immune response to an additional pathogen, such as E. coli or Shigella. For example, the immunogenic composition may include an additional fusion protein, such as ETEC antigens CFA/I/II/IV MEFA, ETEC toxoid fusion 3xSTa_{N12S}-mnLT_{R192G/L211A}, and/or Shigella MEFA. Also disclosed herein are live attenuated bacterial vaccines, including any of the nucleic acids or vectors encoding the fusion proteins disclosed herein expressed in an attenuated bacterial vaccine strain, such as Ty21a.

Also provided are methods of inducing an immune response (such as a protective immune response) in a subject (such as a human subject), including administering any of the fusion proteins, nucleic acids or vectors encoding the fusion protein, pharmaceutical compositions, immunogenic compositions, or live attenuated bacterial vaccines, as described herein. In some examples, the method further includes selecting a subject in or traveling to an area with endemic Vibrio cholerae, and/or selecting a subject that does not have a Vibrio cholerae infection. In some examples, the immunogenic composition is administered subcutaneously (SC), intramuscularly (IM), or intradermally (ID). In some examples, the subject is a human child less than 5 years old.

The foregoing and other features of the disclosure will become more apparent from the following detailed description, which proceeds with reference to the accompanying figures.

### BRIEF DESCRIPTION OF THE DRAWINGS

FIG. 1 is a schematic illustration of an exemplary cholera MEFA genetic structure and epitope sequences.

FIGS. 2A-2C illustrates cholera multi-epitope-fusion-antigen (MEFA) protein construction and characterization. B cell immunodominant epitopes from virulence proteins (cholera toxin (CT) subunit A (CT_{A}) and subunit B (CT_{B}), toxin-coregulated pilus A (TcpA, of O1 El Tor and O1 Classical), sialidase (NanH), flagellin B (FlaB), a conserved epitope for flagellins C and D (FlaC and FlaD), two peptides mimicking O1 lipopolysaccharide O-antigen (LPS), and hemolysin A (HlyA) were in silico identified and integrated into backbone protein flagellin B (FlaB) to construct the cholera MEFA (FIG. 2A). Cholera MEFA protein expressed by pET28a vector in E. coli BL21 (DE3) was extracted and verified in SDS-PAGE with Coomassie blue staining (FIG. 2B) and Western blot with rabbit anti-TcpA antiserum (FIG. 2C).

FIG. 3 shows mouse serum IgG titers (in log₁₀) specific to antigens targeted by the cholera MEFA protein. Serum samples from each mouse in the group IM immunized with cholera MEFA (∇; n=8), cholera MEFA and adjuvant dmLT (∘; n=8), or PBS (+; n=8) were two-fold diluted (1:200 to 1:25,600) and titrated in ELISAs for IgG responses to cholera toxin (CT), toxin-coregulated pilus A (TcpA; O1 classical and O1 El Tor), sialidase, flagellin B (FlaB), flagellin C (FlaC), flagellin D (Fla D), hemolysin A (HlyA), and O1 LPS; HRP-conjugated goat-anti-mouse IgG (1:5,000) as the secondary antibodies. ** and *** indicates a p value of <0.01 and <0.001, respectively, between two groups.

FIG. 4 shows mouse serum antibody adherence inhibition against various Vibrio cholerae strains. Adherence to Caco-2 cells (number of bacterial colony forming units (CFUs), expressed in %; with the number from the control group referred as 100%) by O1 El Tor (N16961), O1 Classical (395), O139 Bengal strain (M045) and non-O1/non-O139 (El Tor 34-D 23) after incubation with the serum samples from mice IM immunized with PBS (white), cholera MEFA protein alone (gray), or cholera MEFA protein and adjuvant dmLT (black). *, **, and *** indicate p values <0.05, <0.01 and <0.001, respectively. Vibrio cholerae O1 El Tor strain N16961, O1 Classical strain 395, O139 strain M045, and non-O1/non-O139 strain El Tor 34-D 23, showed a significant reduction of adherence to Caco-2 cells after incubation with the serum samples of the mice IM immunized with cholera MEFA protein, with or without adjuvant dmLT.

FIGS. 5A-5B show mouse serum antibody neutralization activity against cholera toxin (CT) enterotoxicity and antibody blocking of CT binding of GM₁ receptor. FIG. 5A: Direct cAMP ELISA kit (Enzo Life Sciences) and T-84 cells were used to measure intracellular cyclic AMP levels (nM, pmole/ml) in T-84 cells exposed to CT pre-treated with the serum of the mice IM immunized with cholera MEFA, cholera MEFA adjuvated with dmLT, or the control group (−). Neutralizing antibodies prevent CT from stimulation of cGMP in T-84 cells. Cells treated with culture medium only was to indicate cAMP baseline level, and cells exposed to CT preincubated with PBS (CT+PBS, no serum) as a positive control of CT enterotoxicity stimulation of cGMP. FIG. 5B: Competitive GM₁ ELISA to measure mouse serum antibodies in blocking CT binding to GM₁. Wells coated with GM₁ were incubated with CT mixed with the serum of the mice IM immunized with cholera MEFA protein (▴), cholera MEFA protein and dmLT adjuvant (♦), or PBS the control group (▪). MEFA-induced antibodies that recognize CT B subunits bind to the B subunits, thus block CT from binding to GM₁ receptor, resulting in lower OD values. OD values in the wells incubated with CT mixed with PBS as a positive control to show CT binding to GM₁. ** and *** indicate p values <0.01 and <0.001, respectively.

FIGS. 6A-6C show microscopic images of Vibrio cholerae O1 El Tor N16961 bacterial motility after incubation with PBS (FIG. 6A), sera of the mice IM immunized with PBS (FIG. 6B), or sera of the mice IM immunized with cholera MEFA protein and adjuvant dmLT (FIG. 6C).

FIG. 7 shows adult rabbit serum IgG titers (in log₁₀) specific to the antigens targeted by cholera MEFA protein. Serum samples from the rabbits in the group IM immunized with cholera MEFA (≡; n=4), cholera MEFA and adjuvant dmLT (∘; n=8), or PBS the control group (●; n=8) were two-fold diluted (1:200 to 1:25,600) and titrated for IgG to CT, TcpA (O1 classical), TcpA (O1 El Tor), sialidase, FlaB, FlaC, Fla D and HlyA, with HRP-conjugated goat-anti-rabbit IgG (1:5,000) as the secondary antibodies. ** and *** indicate p values <0.01 and <0.001, respectively.

FIG. 8 shows rabbit fecal IgA titers (in log 2) specific to the antigens targeted by cholera MEFA protein. Cecum content samples from the rabbits in the group IM immunized with cholera MEFA (∇; n=4), cholera MEFA and adjuvant dmLT (∘; n=4), or PBS the control group (●; n=4) were two-fold diluted (1:5 to 1:320) and titrated for IgG to CT, TcpA (O1 classical), TcpA (O1 El Tor), sialidase, FlaB, FlaC, Fla D and HlyA, with HRP-conjugated goat-anti-rabbit IgA (1:3,000) as the secondary antibodies.

FIGS. 9A-9B shows rabbit serum antibody protection against in vitro adherence and in vivo colonization in rabbit small intestines. FIG. 9A: Numbers of O1 El Tor N16961, O1 Classical 395, O139 Bengal and non-O1/non-O139 strain El Tor 34 D23 bacteria (CFUs in %; with the number of bacteria treated with the control serum referred as 100%) adhered to Caco-2 cells after incubation with the serum samples of the adult rabbits IM immunized with PBS the control (white), cholera MEFA protein alone (gray), or cholera MEFA protein and dmLT adjuvant (black). FIG. 9B: The number of V. cholerae O1 or non-O1/non-O139 bacteria (CFUs, per gram rabbit distal ileum) colonized in rabbit small intestine. In one study, rabbits IM immunized with cholera MEFA adjuvanted with dmLT (black; n=4) or PBS control (white; n=4) were orogastrically inoculated with V. cholerae non-O1/non-O139 strain El Tor 34 D-23. In the other study, rabbits were IM immunized with cholera MEFA (gray; n=4), cholera MEFA adjuvanted with dmLT (black; n=4), or PBS control (white; n=4) and then orogastrically inoculated with V. cholerae O1 El Tor N16961 strain. A distal ileal segment was collected from each rabbit at necropsy, ground in PBS (1 gram in 9 ml), and serially diluted. Dilutions were plated on agar plates; bacteria (CFUs) were counted after overnight growth at 30° C. *** indicates a p value of <0.0001.

FIGS. 10A-10D show antigen-specific IgG and IgA responses detected from the pregnant rabbits and born infant rabbits. (FIG. 10A) IgG titers (log₁₀) to cholera toxin (CT), toxin-coregulated pilus A (TcpA; O1 classical or O1 El Tor), sialidase, flagellin B (FlaB), flagellin C (FlaC), flagellin D (Fla D) and hemolysin A (HlyA) detected from serum samples of the pregnant rabbits IM immunized with cholera MEFA protein (adjuvanted with dmLT; grey boxes) (n=7; two rabbits with false pregnancy were included) or the control pregnant rabbits (PBS with dmLT adjuvant; white boxes) (n=6). (FIG. 10B) OD₆₅₀ values of IgG ELISAs detected from the milk samples of the mother rabbits IM immunized with cholera MEFA protein (adjuvanted with dmLT; grey boxes) (n=5) or the control pregnant rabbits (PBS with dmLT adjuvant; white boxes) (n=6). CT, recombinant protein TcpA (O1 classical or O1 El Tor), sialidase, FlaB, FlaC, Fla D or HlyA as the ELISA coating antigen. (FIG. 10C) OD₆₅₀ values of IgA ELISAs detected from the milk samples of the mother rabbits IM immunized with cholera MEFA protein (adjuvanted with dmLT; grey boxes) (n=5) or the control pregnant rabbits (PBS with dmLT adjuvant; white boxes) (n=6). (FIG. 10D) IgG titers (log₁₀) to CT, TcpA (O1 classical or O1 El Tor), sialidase, FlaB, FlaC, Fla D and HlyA detected from the serum samples of each infant rabbits born to mothers IM immunized with cholera MEFA protein and adjuvant dmLT (n=34; grey boxes) or the control mothers immunized with PBS and dmLT adjuvant (n=40; white boxes). No anti-O1 LPS antibody responses were detected from the mother rabbits or infant rabbits. * indicates a p value of <0.05, and *** a p value of <0.0001.

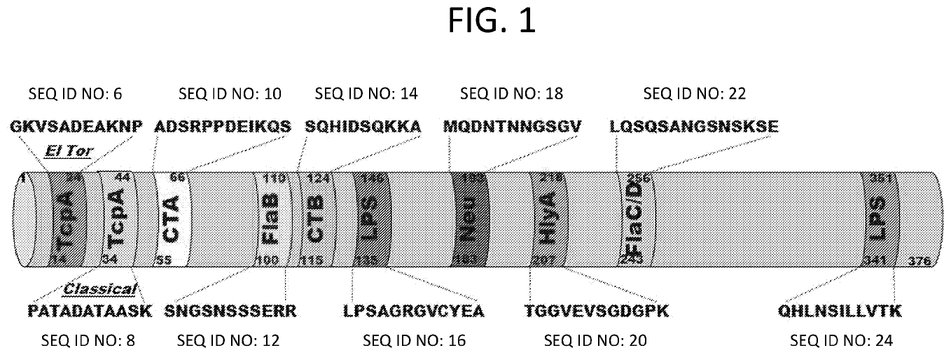

### SEQUENCE LISTING

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand. The Sequence Listing is submitted as an ASCII text file (Sequence_listing.txt), created on Mar. 17, 2022, 32,768 bytes, which is incorporated by reference herein. In the accompanying sequence listing: SEQ ID NO: 1 is an exemplary nucleic acid sequence encoding a cholera MEFA nucleotide sequence containing TcpA (O1 El Tor biotype), TcpA (O1 Classical biotype), CTA, FlaB, CTB, LPS (SEQ ID NO: 16), Neu, HlyA, FlaC/D, and LPS (SEQ ID NO: 24) epitopes. Bold sequences indicate sequences encoding epitopes.

ATGGGCGCGATTAACGTGAACACCAACGTGAGCGCGATGACCGGCAAAGT
GAGCGCGGATGAAGCGAAAAACCCGATGCAGAAAAGCATGGAACGCCTGA
GCCCGGCGACCGCGGATGCGACCGCGGCGAGCAAAGCGGCGGGCCTGCAG
ATTAGCAACCGCCTGGCGGATAGCCGCCCGCCGGATGAAATTAAACAGAG
CAACGCGAACGATGGCATTAGCATTGCGCAGACCGCGGAAGGCGCGATGA
ACGAAACCACCAACATTCTGCAGCGCATGCGCGATCTGGCGCTGCAGAGC
AGCAACGGCAGCAACAGCAGCAGCGAACGCCGCGCGATTCAGGAAAGCCA
GCATATTGATAGCCAGAAAAAAGCGCGCATTGCGGAAACCACCAGCTTTG
GCGGCCTGCCGAGCGCGGGCCGCGGCGTGTGCTATGAAGCGTTTCAGATT
GGCGCGGATAGCGGCGAAGCGGTGATGCTGAGCATGGGCAGCATGCGCAG
CGATACCCAGGCGATGGGCGGCAAAAGCTATCGCGCGCAGGAAGGCAAAA
TGCAGGATAACACCAACAACGGCAGCGGCGTGCTGACCCTGAGCTATACC
AACAAACAGGGCGAAGCGCGCACCGGCGGCGTGGAAGTGAGCGGCGATGG
CCCGAAAGAAGAACTGGCGACCTATATTAACGGCCAGACCGAAGATGTGA
AAGCGAGCGTGGGCGAAGATGGCAAACTGCTGCAGAGCCAGAGCGCGAAC
GGCAGCAACAGCAAAAGCGAAATTGGCGGCGGCCTGGGCGGCGAAATTGG
CTTTGATGCGGGCCGCAACGTGACCGTGGCGGATGTGAACGTGAGCACCG
TGGCGGGCAGCCAGGAAGCGGTGAGCATTCTGGATGGCGCGCTGAAAGCG
GTGGATAGCCAGCGCGCGAGCCTGGGCGCGTTTCAGAACCGCTTTGGCCA
TGCGATTAGCAACCTGGATAACGTGAACGAAAACGTGAACGCGAGCCGCA
GCCGCATTCGCGATACCGATTATCAGCATCTGAACAGCATTCTGCTGGTG
ACCAAAATTCTGCAGCAGGCGAGCACCAGCGTGCTGGCGCAGGCGAAACA
GAGCCCGAGCGCGGCGCTGAGCCTGCTGGGCTAA

SEQ ID NO: 2 is an exemplary cholera MEFA amino acid sequence containing TcpA (O1 El Tor biotype), TcpA (O1 Classical biotype), CTA, FlaB, CTB, LPS (SEQ ID NO: 15), Neu, HlyA, FlaC/D, and LPS (SEQ ID NO: 23) epitopes. Bold sequences indicate epitope amino acid sequences.

MGAINVNTNVSAMTGKVSADEAKNPMQKSMERLSPATADATAASKAAGLQ
ISNRLADSRPPDEIKQSNANDGISIAQTAEGAMNETTNILQRMRDLALQS
SNGSNSSSERRAIQESQHIDSQKKARIAETTSFGGLPSAGRGVCYEAFQI
GADSGEAVMLSMGSMRSDTQAMGGKSYRAQEGKMQDNTNNGSGVLTLSYT
NKQGEARTGGVEVSGDGPKEELATYINGQTEDVKASVGEDGKLLQSQSAN
GSNSKSEIGGGLGGEIGFDAGRNVTVADVNVSTVAGSQEAVSILDGALKA
VDSQRASLGAFQNRFGHAISNLDNVNENVNASRSRIRDTDYQHLNSILLV
TKILQQASTSVLAQAKQSPSAALSLLG

SEQ ID NO: 3 is an exemplary consensus nucleic acid sequence that encodes a backbone protein, where ‘N’ indicates a location for insertion or substitution of an epitope. The indicated size of the insertion or substitution is not limiting, and can be varied.

ATGGGCGCGATTAACGTGAACACCAACGTGAGCGCGATGACCNNNNNNNN
NNNNNNNNNNNNNNNNNNNNNNNNNATGCAGAAAAGCATGGAACGCCTGA
GCNNNNNNNNNNNNNNNNNNNNNNNNNNNNNNNNNGCGGCGGGCCTGCAG
ATTAGCAACCGCCTGNNNNNNNNNNNNNNNNNNNNNNNNNNNNNNNNNNN
NAACGCGAACGATGGCATTAGCATTGCGCAGACCGCGGAAGGCGCGATGA
ACGAAACCACCAACATTCTGCAGCGCATGCGCGATCTGGCGCTGCAGAGC
NNNNNNNNNNNNNNNNNNNNNNNNNNNNNNNNNGCGATTCAGGAANNNNN
NNNNNNNNNNNNNNNNNNNNNNNNNCGCATTGCGGAAACCACCAGCTTTG
GCGGCNNNNNNNNNNNNNNNNNNNNNNNNNNNNNNNNNNNNTTTCAGATT
GGCGCGGATAGCGGCGAAGCGGTGATGCTGAGCATGGGCAGCATGCGCAG
CGATACCCAGGCGATGGGCGGCAAAAGCTATCGCGCGCAGGAAGGCAAAN
NNNNNNNNNNNNNNNNNNNNNNNNNNNNNNNNCTGACCCTGAGCTATACC
AACAAACAGGGCGAAGCGCGCNNNNNNNNNNNNNNNNNNNNNNNNNNNNN
NNNNNNNGAAGAACTGGCGACCTATATTAACGGCCAGACCGAAGATGTGA
AAGCGAGCGTGGGCGAAGATGGCAAACTGNNNNNNNNNNNNNNNNNNNNN
NNNNNNNNNNNNNNNNNNNNNATTGGCGGCGGCCTGGGCGGCGAAATTGG
CTTTGATGCGGGCCGCAACGTGACCGTGGCGGATGTGAACGTGAGCACCG
TGGCGGGCAGCCAGGAAGCGGTGAGCATTCTGGATGGCGCGCTGAAAGCG
GTGGATAGCCAGCGCGCGAGCCTGGGCGCGTTTCAGAACCGCTTTGGCCA
TGCGATTAGCAACCTGGATAACGTGAACGAAAACGTGAACGCGAGCCGCA
GCCGCATTCGCGATACCGATTATNNNNNNNNNNNNNNNNNNNNNNNNNNN
NNNNNNATTCTGCAGCAGGCGAGCACCAGCGTGCTGGCGCAGGCGAAACA
GAGCCCGAGCGCGGCGCTGAGCCTGCTGGGCTAA

SEQ ID NO: 4 is an exemplary consensus amino acid sequence for backbone protein, where ‘X’ indicates a location for insertion or substitution of an epitope. The indicated size of the insertion or substitution is not limiting, and can be varied.

MGAINVNTNVSAMTXXXXXXXXXXXMQKSMERLSXXXXXXXXXXXAAGL
QISNRLXXXXXXXXXXXXNANDGISIAQTAEGAMNETTNILQRMRDLAL
QSXXXXXXXXXXXAIQEXXXXXXXXXXRIAETTSFGGXXXXXXXXXXXX
FQIGADSGEAVMLSMGSMRSDTQAMGGKSYRAEQEGKXXXXXXXXXXXL
TLSYTNKQEGEARXXXXXXXXXXXXEELATYINGQTEDVKASVGEDGKL
XXXXXXXXXXXXXXIGGGLGGEIGFDAGRNVTVADVNVSTVAQSQEAVS
ILDGALKAVDSQRASLGAFQNRFGHAISNLDNVNENVNASRSRIRDTDY
XXXXXXXXXXXILQQASTSVLAQAKQSPSAALSLLG

SEQ ID NO: 5 is an exemplary nucleic acid sequence encoding a TcpA epitope from O1 El Tor biotype. GGCAAAGTGAGCGCGGATGAAGCGAAAAACCCG

SEQ ID NO: 6 is an exemplary TcpA epitope amino acid sequence from O1 El Tor biotype. GKVSADEAKNP

SEQ ID NO: 7 is an exemplary nucleic acid sequence encoding a TcpA epitope from O1 classical biotype. CCGGCGACCGCGGATGCGACCGCGGCGAGCAAA

SEQ ID NO: 8 is an exemplary TcpA epitope amino acid sequence from O1 classical biotype. PATADATAASK

SEQ ID NO: 9 is an exemplary nucleic acid sequence encoding an CTA epitope.

GCGGATAGCCGCCCGCCGGATGAAATTAAACAGAGC

SEQ ID NO: 10 is an exemplary CTA epitope amino acid sequence. ADSRPPDEIKQS

SEQ ID NO: 11 is an exemplary nucleic acid sequence encoding a FlaB epitope.

AGCAACGGCAGCAACAGCAGCAGCGAACGCCGC

SEQ ID NO: 12 is an exemplary FlaB epitope amino acid sequence. SNGSNSSSERR

SEQ ID NO: 13 is an exemplary nucleic acid sequence encoding a CTB epitope.

AGCCAGCATATTGATAGCCAGAAAAAAGCG

SEQ ID NO: 14 is an exemplary CTB epitope amino acid sequence. SQHIDSQKKA

SEQ ID NO: 15 is an exemplary nucleic acid sequence encoding a LPS O-antigen mimic (LPS) epitope.

SEQ ID NO: 16 is an exemplary LPS O-antigen mimic (LPS) epitope amino acid sequence.

LPSAGRGVCYEA

SEQ ID NO: 17 is an exemplary nucleic acid sequence encoding a sialidase (Neu) epitope.

ATGCAGGATAACACCAACAACGGCAGCGGCGTG

SEQ ID NO: 18 is an exemplary sialidase (Neu) epitope amino acid sequence.

MQDNTNNGSGV

SEQ ID NO: 19 is an exemplary nucleic acid sequence encoding a HlyA epitope.

ACCGGCGGCGTGGAAGTGAGCGGCGATGGCCCGAAA

SEQ ID NO: 20 is an exemplary HlyA epitope amino acid sequence. TGGVEVSGDGPK

SEQ ID NO: 21 is an exemplary nucleic acid sequence encoding a FlaC/D epitope.

CTGCAGAGCCAGAGCGCGAACGGCAGCAACAGCAAAAGCGAA

SEQ ID NO: 22 is an exemplary FlaC/D epitope amino acid sequence.

LQSQSANGSNSKSE

SEQ ID NO: 23 is an exemplary nucleic acid sequence encoding a further LPS O-antigen mimic (LPS) epitope. CAGCATCTGAACAGCATTCTGCTGGTGACCAAA

SEQ ID NO: 24 is an exemplary further LPS O-antigen mimic (LPS) epitope amino acid sequence. QHLNSILLVTK

SEQ ID NO: 25 is an exemplary nucleic acid encoding a fusion protein with ETEC antigens (CFA/I/II/IV MEFA).

ATGGAAATGGCTAGCGCAGTAGAGGATTTTTTCATTGTTCCAGTTTCTGG
AGATCCTGCAATTGATCTTTTGCAAGCTGATGGCAATGCTCTGCCATCAG
CTGTAAAGTTAGCTTATTCTCCCGCATCAAAAACTAATACTTTGGTGGGT
GTTTTGACTCTTGTACATACAAACGATGCAACTAAAAAAAATGTACTAGT
TAAGCTTGTAACACCACAGCTTACAGATGTTCTGAATCCAACCCTGCAAA
TTCCTGTTTCTGTGCAGGTAACGGTCTACCCTGTTTCTACAACAGCCAAA
GAATTTGAAGCTGCTGCTTTGGGATATTCTGCATCCGGTGTAAATGGCTT
GGTGTCAATTGTGCTTACTGTAATTAGCGCTGCACCTAAAACTGCCGGTA
CCGCCCCAACTGCAGGAAACTATTCAGGAGTAGTATCTCTTGTAATGACT
TTGGGAGCCTGATAACGGCCGCACTCGAGC

SEQ ID NO: 26 is an exemplary fusion protein with ETEC antigens (CFA/I/II/IV MEFA).

MEMASAVEDFFIVPVSGDPAIDLLQADGNALPSAVKLAYSPASKTNTLVG
VLTLVHTNDATKKNVLVKLVTPQLTDVLNPTLQIPVSVQVTVYPVSTTAK
EFEAAALGYSASGVNGLVSIVLTVISAAPKTAGTAPTAGNYSGVVSLVMT
LGA

SEQ ID NO: 27 is an exemplary nucleic acid encoding a fusion protein with ETEC toxoid antigen (toxoid fusion 3xSTa_{N12S}-mnLT_{R192G/L211A}).

ATGGAAATGGCTAGCATGAATAGTAGCAATTACTGCTGTGAATTGTGTTG
TAGCCCTGCTTGTACCGGGTGCTATGGGCCGGGGCCCAATGGCGACAAAT
TATACCGTGCTGACTCTAGACCCCCAGATGAAATAAAACGTTCCGGAGGT
CTTATGCCCAGAGGGCATAATGAGTACTTCGATAGAGGAACTCAAATGAA
TATTAATCTTTATGATCACGCGAGAGGAACACAAACCGGCTTTGTCAGAT
ATGATGACGGATATGTTTCCACTTCTCTTAGTTTGAGAAGTGCTCACTTA
CGAGGACAGTCTATATTATCAGGATATTCCACTTACTATATATATGTTAT
AGCGACAGCACCAAATATGTTTAATGTTAATGATGTATTAGGCGTATACA
GCCCTCACCCATATGAACAGGAGGTTTCTGCGTTAGGTGGAATACCATAT
TCTCAGATATATGGATGGTATCGTGTTAATTTTGGTGTGATTGATGAACG
ATTACATCGTAACAGGGAATATAGAGACCGGTATTACAGAAATCTGAATA
TAGCTCCGGCAGAGGATGGTTACAGATTAGCAGGTTTCCCACCGGATCAC
CAAGCTTGGAGAGAAGAACCCTGGATTCATCATGCACCACAAGGTTGTGG
AAATTCATCAGGAGGGCCGGTCGACATGAATAGTAGCAATTACTGCTGTG
AATTGTGTTGTAGCCCTGCTTGTACCGGGTGCTATACAATTACAGGTGAT
ACTTGTAATGAGGAGACCCAGAATCTGAGCACAATATATGCCAGGAAATA
TCAATCAAAAGTTAAGAGGCAGATATTTTCAGACTATCAGTCAGAGGTTG
ACATATATAACAGAATTCGGAATGAATTAGGGCCGGGGCCCGCTCCCCAG
TCTATTACAGAACTATGTTCGGAATATCGCAACACACAAATATATACGAT
AAATGACAAGATACTATCATATACGGAATCGATGGCAGGCAAAAGAGAAA
TGGTTATCATTACATTTAAGAGCGGCGCAACATTTCAGGTCGAAGTCCCG
GGCAGTCAACATATAGACTCCCAAAAAAAAGCCATTGAAAGGATGAAGGA
CACATTAAGAATCACATATCTGACCGAGACCAAAATTGATAAATTATGTG
TATGGAATAATAAAACCCCCAATTCAATTGCGGCAATCAGTGATCCCCGG
GTACCGAGCTCGATGAATAGTAGCAATTACTGCTGTGAATTGTGTTGTAG
CCCTGCTTGTACCGGGTGCTATTAATAA

SEQ ID NO: 28 is an exemplary fusion protein with ETEC toxoid antigen (toxoid fusion 3xSTa_{N12S}-mnLT_{R192G/L211A}).

MEMASMNSSNYCCELCCSPACTGCYGPGPNGDKLYRADSRPPDEIKRSGG
LMPRGHNEYFDRGTQMNINLYDHARGTQTGFVRYDDGYVSTSLSLRSAHL
AGQSILSGYSTYYIYVIATAPNMFNVNDVLGVYSPHPYEQEVSALGGIPY
SQIYGWYRVNFGVIDERLHRNREYRDRYYRNLNIAPAEDGYRLAGFPPDH
QAWREEPWIHHAPQGCGNSSGGPVDMNSSNYCCELCCSPACTGCYTITGD
TCNEETQNLSTIYARKYQSKVKRQIFSDYQSEVDIYNRIRNELGPGPAPQ
SITELCSEYRNTQIYTINDKILSYTESMAGKREMVIITFKSGATFQVEVP
GSQHIDSQKKAIERMKDTLRITYLTETKIDKLCVWNNKTPNSIAAISDPR
VPSSMNSSNYCCELCCSPACTGCY

SEQ ID NO: 29 is an exemplary nucleic acid sequence encoding a fusion protein with a Shigella IpaD backbone protein and homologous and heterologous epitopes from Shigella virulence factors IpaD, IpaB, VirG, GuaB, StxA, Stx2A, and StxB.

ATGGGCAATATAACAACTCTGACTAATAGTATTTCCACCTCATCATTCAG
CCCAAACAATACCAACGGTTCATCAACCGAAACAGTTAATTCTGATATAA
AAACAACGACCAGTTCTCATCCTGTAAGTTCCCTTACTATGCTCAACGAC
ACCCTTCATAATATCAGAACAACAAATCAGGCATTAAAGAAAGATCTCTC
ACAAAAAACGTTGACTAAAACATCGCTAGAAGAAATAGCATTACATTCAT
CTCAGATTAGCATGGATGTAAACAAATCCGCTCAACTATTGGATATTCTT
TCCAAGAAAGAATATCCAATTAATAAAGACGCAAGAGAATTATTACATTC
AGCCCAACTAGTTGGAAAAAATAATGAAGAATCTATGATATCTCACAGAG
AACTGTGGGATAAAATTGCAAAGTCAATCAATAATATTAATGAACAGTAT
CTGAAAGTATATGAACATGCCGTTAGTTCATATACTCAATCTGCTGAACA
GCTATCAACCCAGCAGAAAAGTCTTGCCGGCTGGATCTCTCCCGGAGGTA
ACGACGGAAACTCCGTGAAATTACAAGTCAAGTCTCTTAAAAGTGATAGT
GATGGGGGGAATGGAGGTGATAAAGATAAACCGCTATATCCAGCAAATAA
TACTGTTCTGCCTGACTATCATGGACAAGACTCTGTTCGTGTAGAATTAG
GTGGAACAGAACGTAAACCGAACGCTTGTAAAGACGAGTATGTTGTCAAT
ATAAACATGACCCCAATAGACAATATGCTTCCGGAGTATCGGGGAGAGGA
TGGTGTCAGAGTGGGTGAGGTTGTGCTAGATAATGCAAAATATCAGGCAT
GGAATGCCGGAGAGTATACAAAATATAATGATGACGATACCAATCTTCAA
ACCTTAGTTCAAAAATACAGTAATGCCAATAGTATTTTTGATAATTTAGT
AAAGGTTTTGAGTAGTACAATAAGTTCATGTACAGATACAGATAAACTTT
TTCTCCATTTCTGA

SEQ ID NO: 30 is an exemplary fusion protein with a Shigella IpaD backbone protein and homologous and heterologous epitopes from Shigella virulence factors IpaD, IpaB, VirG, GuaB, StxA, Stx2A, and StxB.

MGNITTLTNSISTSSFSPNNINGSSTETVNSDIKTTTSSHPVSSLTMLND
TLHNIRTTNQALKKDLSQKTLTKTSLEEIALHSSQISMDVNKSAQLLDIL
SKKEYPINKDARELLHSAQLVGKNNEESMISHRELWDKIAKSINNINEQY
LKVYEHAVSSYTQSAEQLSTQQKSLAGWISPGGNDGNSVKLQVKSLKSDS
DGGNGGDKDKPLYPANNTVLPDYHGQDSVRVELGGTERKPNACKDEYVVN
INMTPIDNMLPEYRGEDGVRVGEVVLDNAKYQAWNAGEYTKYNDDDTNLQ
TLVQKYSNANSIFDNLVKVLSSTISSCTDTDKLFLHF

SEQ ID NO: 31 is an exemplary further LPS O-antigen mimic (LPS) epitope amino acid sequence. CFFPNLSYC

SEQ ID NO: 32 is an exemplary further LPS O-antigen mimic (LPS) epitope amino acid sequence. SMCMHGGAYCFP

SEQ ID NOS: 33-46 are exemplary primers.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising an epitope” includes single or plural epitopes and is considered equivalent to the phrase “comprising at least one epitope.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided.

Adjuvant: A substance or vehicle that non-specifically enhances the immune response to an antigen (for example, a Vibrio cholerae antigen). Adjuvants can be used with the compositions disclosed herein, for example, as part of a Vibrio cholerae immunogenic composition or pharmaceutical composition provided herein. Adjuvants can include a suspension of minerals (alum, aluminum hydroxide, or phosphate) on which an antigen is adsorbed or a water-in-oil emulsion in which an antigen solution is emulsified in mineral oil (for example, Freund's incomplete adjuvant), sometimes with the inclusion of killed mycobacteria (Freund's complete adjuvant) to further enhance antigenicity. Immunostimulatory oligonucleotides (such as those including a CpG motif) can also be used as adjuvants (for example, see U.S. Pat. Nos. 6,194,388; 6,207,646; 6,214,806; 6,218,371; 6,239,116; 6,339,068; 6,406,705; and 6,429,199). Adjuvants may also include biological molecules, such as costimulatory molecules. Exemplary biological adjuvants include IL-2, RANTES, GM-CSF, TNF-α, IFN-γ, G-CSF, LFA-3, CD72, B7-1, B7-2, OX-40L and 41 BBL. In one example the adjuvant is one or more toll-like receptor (TLR) agonist, such as an agonist of TLR1/2 (which can be a synthetic ligand) (for example, Pam3Cys), TLR2 (for example, CFA, Pam2Cys), TLR3 (for example, polyL:C, poly A:U), TLR4 (for example, MPLA, Lipid A, and lipopolysaccharide), TLR5 (for example, flagellin), TLR7 (for example, gardiquimod, imiquimod, loxoribine, Resiquimod®), TLR7/8 (for example, R0848), TLR8 (for example, imidazoquionolines, ssPolyU, 3M-012), TLR9 (for example, ODN 1826 (type B), ODN 2216 (type A), CpG oligonucleotides) and/or TLR11/12 (for example, profilin). In one example, the adjuvant is lipid A, such as lipid A monophosphoryl (MPL) from Salmonella enterica serotype Minnesota Re 595 (for example, Sigma Aldrich Catalog #L6895). In another example the adjuvant is an enterotoxin-based adjuvant, such as double mutant heat-labile toxin (dmLT).

Antigen or immunogen: A composition that can stimulate the production of an immune response in a subject, including compositions that are injected or absorbed into a subject. An antigen reacts with the products of specific humoral or cellular immunity, including those induced by heterologous immunogens.

Attenuated: In the context of the type of live pathogen, a pathogen (for example, a bacterial pathogen, such as Ty21a) is attenuated if its ability to produce disease is reduced (or even eliminated) compared with a wild-type pathogen.

Cholera: A disease caused by Vibrio cholerae infection, characterized by severe, dehydrating diarrhea. Cholera remains a persistent cause of morbidity and mortality in Asia and Africa. Globally, there are 1.3 to 4 million cholera cases with 21,000 to 143,000 deaths annually. Young children are particularly vulnerable, as current oral vaccines have shown poor efficacy in children under five in regions with endemic cholera.

Epitope: The portion of an antigen that is recognized by an antibody or antigen receptor. Epitopes are also known as antigenic determinants. In some examples, the epitope is a peptide sequence from a Vibrio cholerae protein, such as one or more of: toxin coregulated pilus A subunit (TcpA), cholera toxin (CTA), flagellin B subunit (FlaB), cholera toxin subunit B (CTB), sialidase (Neu; also referred to as NanH), hemolysin A (HlyA), and flagellin C (FlaC), and flagellin D (FlaD), or a synthetic peptide that mimics one or more polysaccharide Vibrio cholerae epitopes (also known as “mimotopes”), for example, an O1 lipopolysaccharide O-antigen mimic (LPS) (Ghazi and Gargari (2017) Iranian Journal of Microbiology 9:244-250; Dharmasena et al. (2009) Microbiol. 155:2353-2364; Falklind-Jerkerus et al., (2005) Microbes Infect. 7:1453-1460).

Escherichia coli (E. coli): E. coli is a Gram-negative, facultative anaerobic, rod-shaped, coliform bacterium commonly found in the lower intestine of warm-blooded organisms (endotherms). Virulent strains can cause serious disease. In specific examples, the E. coli is enterotoxigenic E. coli (ETEC), which is the most common cause of traveler's diarrhea with 840 million cases worldwide in developing countries each year. The bacteria are typically transmitted through contaminated food or drinking water, adhere to the intestinal lining, where they secrete enterotoxins, leading to watery diarrhea. The rate and severity of infections are higher among children under the age of five, which include 380,000 deaths annually. Administration of antibiotics has been shown to shorten the course of illness and duration of excretion of ETEC in adults in endemic areas and in traveler's diarrhea, but the rate of resistance to commonly used antibiotics is increasing and they are generally not recommended. The antibiotic used depends upon susceptibility patterns in the particular geographical region; the antibiotics typically selected for administration include fluoroquinolones, azithromycin, and rifaximin.

Fusion protein: A protein containing amino acid sequence from at least two different (heterologous) proteins or peptides. In some examples herein, the fusion protein includes a backbone protein and one or more heterologous peptides, such as one or more heterologous epitopes. The backbone and heterologous sequences may be contiguous or non-contiguous. Fusion proteins can be generated, for example, by expression of a nucleic acid sequence engineered from nucleic acid sequences encoding at least a portion of two different (heterologous) proteins. To create a fusion protein, the nucleic acid sequences must be in the same reading frame and contain no internal stop codons. Fusion proteins, particularly short fusion proteins, can also be generated by chemical synthesis.

Heterologous: A heterologous protein or nucleic acid refers to a protein or nucleic acid derived from a different source (such as a peptide or epitope from a different protein).

Homologous: A homologous protein or nucleic acid refers to a protein or nucleic acid derived from the same source (such as a peptide or epitope from the same protein).

Immune response: A response of a cell of the immune system, such as a B-cell, T-cell, macrophage, or polymorphonucleocyte, to a stimulus such as an antigen/immunogen or vaccine (such as a Vibrio cholerae immunogenic composition or vaccine). An immune response can include any cell of the body involved in a host defense response, including for example, an epithelial cell that secretes an interferon or a cytokine. An immune response includes, but is not limited to, an innate immune response. As used herein, a protective immune response refers to an immune response that protects a subject from infection (prevents infection or prevents the development of disease associated with infection). Methods of measuring immune responses include, for example, measuring proliferation and/or activity of lymphocytes (such as B or T cells), secretion of cytokines or chemokines, inflammation, antibody production and the like.

Isolated: An “isolated” biological component (such as a nucleic acid, protein, or cell) has been substantially separated or purified away from other biological components (such as cell debris, or other proteins or nucleic acids). Biological components that have been “isolated” include those components purified by standard purification methods. The term also embraces recombinant nucleic acids and proteins and chemically synthesized nucleic acids or peptides.

Modified protein or nucleic acid: A nucleic acid or protein having one or more changes in sequence. For example, sequence modifications include substitutions, insertions, and deletions, or combinations thereof. For proteins, insertions include amino and/or carboxyl terminal fusions as well as intrasequence insertions of single or multiple amino acid residues. Insertions for nucleic acid sequence include 5′ or 3′ additions or intrasequence insertions of single or multiple nucleotides. Deletions are characterized by the removal of one or more amino acid residues from a protein sequence or one or more nucleotides from a nucleic acid sequence. Substitutions are those in which at least one amino acid residue or nucleotide has been removed and a different residue or nucleotide inserted in its place.

Substitutions, deletions, insertions or any combination thereof may be combined to arrive at a final modified sequence. Protein modifications can be prepared by modification of nucleotides in the DNA encoding the protein, thereby producing DNA encoding the modification. Techniques for making insertion, deletion and substitution modifications at predetermined sites in DNA or RNA having a known sequence are known.

Operably linked: A first nucleic acid or amino acid sequence is operably linked with a second nucleic acid or amino acid sequence when the first sequence is placed in a functional relationship with the second sequence. In one example, a promoter is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: Pharmaceutically acceptable carriers have been described, for example, Remington's Pharmaceutical Sciences, by E. W. Martin, Mack Publishing Co., Easton, PA, 22nd Edition, 2013, describes compositions and formulations suitable for pharmaceutical delivery of the disclosed compositions.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), non-toxic solid carriers can include, for example, pharmaceutical grades of mannitol, lactose, starch, or magnesium stearate. In addition to biologically neutral carriers, pharmaceutical compositions (such as immunogenic compositions) to be administered can contain minor amounts of non-toxic auxiliary substances, such as wetting or emulsifying agents, preservatives, and pH buffering agents, and the like, for example sodium acetate or sorbitan monolaurate. In particular embodiments, in compositions suitable for administration to a subject, the carrier may be sterile and/or suspended or otherwise contained in a unit dosage form containing one or more measured doses of the composition suitable to induce the desired immune response. The unit dosage form may be, for example, in a sealed vial that contains sterile contents or a syringe for injection into a subject, or lyophilized for subsequent solubilization and administration, or in a solid or controlled release dosage.

Preventing, treating or ameliorating a disease: “Preventing” a disease (such as cholera) refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Recombinant: A recombinant nucleic acid molecule is one that has a sequence that is not naturally occurring, for example, a nucleic acid that includes one or more substitutions, deletions, or insertions, and/or has a sequence that is made by an artificial combination of two otherwise separated segments of sequence. This artificial combination can be accomplished by chemical synthesis or by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In some embodiments, a recombinant protein includes a fusion protein, for example, a protein that includes one or more heterologous peptides (such as epitopes).

Subject: Living multi-cellular vertebrate organisms, a category that includes both human and non-human mammals. In some examples, a subject is one that can be infected with Vibrio cholerae, such as humans.

Therapeutically effective amount (or effective amount): The amount of an agent or composition, such as a disclosed fusion protein and compositions thereof, that is sufficient to induce a desired response, such as an immune response, or is sufficient to treat, reduce, and/or ameliorate the symptoms and/or underlying causes of a disorder or disease/In some embodiments, a therapeutically effective amount is an amount that induces an immune response to Vibrio cholerae or inhibits and/or treats Vibrio cholerae infection and/or disease resulting therefrom (such as cholera). In some embodiments, a therapeutically effective amount is an amount sufficient to reduce or eliminate a symptom of a disease (such as cholera). In some examples, it is an amount effective to inhibit pathogen (such as Vibrio cholerae) replication, motility, ability to infect host cells, or otherwise measurably alter outward symptoms of pathogen infection.

In one example, a desired response is to inhibit or reduce Vibrio cholerae infection. Vibrio cholerae infection does not need to be completely eliminated for the method to be effective. For example, administration of a therapeutically effective amount of the agent can decrease the Vibrio cholerae (for example, as measured by infection of cells, or by number or percentage of subjects infected by Vibrio cholerae) by a desired amount, for example by at least 50%, at least 60%, at least 70%, at least 80%, at least 90%, at least 95%, at least 98%, or even at least 99% compared to a suitable control.

It is understood that producing a protective immune response against a pathogen can require multiple administrations of the immunogenic composition. Thus, a therapeutically effective amount encompasses a fractional dose that contributes in combination with previous or subsequent administrations to attain a protective immune response. For example, a therapeutically effective amount of an agent can be administered in a single dose, or in several doses, during a course of treatment (such as a prime-boost vaccination treatment). However, the therapeutically effective amount and timing of administration can depend on the subject being treated, the severity and type of the condition being treated, and the manner of administration. A unit dosage form of the agent can be packaged in a therapeutic amount, or in multiples of the therapeutic amount, for example, in a vial (such as with a pierceable lid) or syringe having sterile components.

Vaccine: A preparation of immunogenic material capable of stimulating an immune response. The immunogenic material may include attenuated or killed microorganisms (such as attenuated viruses or bacteria) or antigenic proteins, peptides, or nucleic acids encoding an antigen.

Vaccines may elicit both prophylactic and therapeutic responses. Methods of administration vary according to the vaccine, but may include inoculation, ingestion, inhalation, or other forms of administration. Inoculations can be delivered by any of a number of routes, including oral or parenteral, such as intravenous, subcutaneous, or intramuscular. In specific embodiments, vaccines can be administered via an intramuscular route. Vaccines may be administered with an adjuvant to increase the immune response to the vaccine.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic nucleic acids and/or selectable marker genes and other genetic elements. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like. Exemplary vectors include plasmids, viral vectors, cosmids, and artificial chromosomes. In some examples, the vector is a viral vector such as an adeno-associated vector (AAV) or lentiviral vector.

Vibrio cholerae (V. cholerae): A Gram-negative, halophilic and highly motile curved-rod bacterium. Vibrio cholerae naturally occurs in aquatic environments. A subset of Vibrio cholerae strains, which carry the genes for cholera toxin (CT) and the Vibrio pathogenicity island (VPI), cause cholera, a disease characterized by severe, dehydrating diarrhea. Nearly all epidemic strains are either O group 1 (O1) or O group 139 (O139), however, non-O1/non-O139 (e.g. O3, O37, O75, O141) serogroup strains also cause severe gastrointestinal infections sporadically. Vibrio cholerae O1 is further divided into two biotypes, classical and El Tor, which differ in the severity of clinical symptoms and the expression and regulation of major virulence factors. Vibrio cholerae is typically contracted by ingesting contaminated water or food (usually fecal contamination from an individual infected with Vibrio cholerae), or by ingesting raw or undercooked shellfish. More information on Vibrio cholerae pathogenesis can be found, for example, in Silva and Benitez (2016) PLoS Negl Trop Dis. 10(2):e0004330.

Virulence factors: Virulence factors enable bacteria to replicate and disseminate within a host, in part by subverting or eluding host defenses. In some embodiments, virulence factors include Vibrio cholerae virulence factors, such as flagellin B subunit (FlaB), toxin coregulated pilus A (TcpA), cholera toxin (CTA), cholera toxin subunit B (CTB), sialidase (Neu; also called NanH), hemolysin A (HlyA), flagellin C (FlaC), or flagellin D (FlaD) as well as epitopes mimicking O1 LPS O-antigen domains (LPS) that mimic native antigenicity.

### Overview

### I. Fusion Proteins

Disclosed herein are fusion proteins including one or more heterologous Vibrio cholerae virulence factor epitope. In some examples, the fusion protein includes at least one backbone protein with a consensus sequence that has at least 90% sequence identity to SEQ ID NO: 4. In some examples, the fusion protein includes at least one heterologous Vibrio cholerae epitope, such as a peptide of a Vibrio cholerae virulence factor. In further examples, the fusion protein includes at least one homologous epitope, such as a FlaB epitope. In specific, non-limiting examples, the fusion protein has at least 90% sequence identity to SEQ ID NO: 2, or includes or consists of SEQ ID NO: 2.

### Backbone Proteins

The disclosed fusion proteins include at least one backbone protein. One or more backbone proteins can be combined into one fusion protein, for example, by using a linker peptide. In some examples, the fusion protein comprises at least 1, at least 2, at least 3, at least 4, or at least 5 backbone proteins. In some examples, the at least one backbone protein allows the fusion protein to maintain a tertiary or quaternary structure while presenting one or more epitopes (such as at least one heterologous epitope and/or homologous epitope). In some examples, the backbone protein includes a series of peptides from a single protein or protein fold, for example, a backbone protein can include the same or similar tertiary or quaternary structural features as a single protein or protein fold. Example structural features that a backbone protein can retain from a single protein or protein fold include solvent inaccessible amino acids and interactions, salt bridges, disulfide bonds, secondary structure, and/or protein fold. In some examples, the backbone protein is derived from a Vibrio cholerae virulence factor. In specific examples, the backbone protein is derived from the Vibrio cholerae virulence factor flagellin B subunit (FlaB) of Vibrio cholerae O1 serotype.

An exemplary consensus nucleic acid sequence encoding a backbone protein is provided as SEQ ID NO: 3. ‘N’ indicates a location for insertion or substitution with a heterologous or homologous epitope. The number of “Ns” at a particular insertion or substitution site is not determinative of the length of an epitope to be inserted. Rather, the number of nucleotides (‘N’) at each epitope site will vary depending on the length of the inserted epitope. A plurality of “N” is merely provided for ease of identification of each insertion site in SEQ ID NO: 3. Thus, the inserted or substituted epitope may be any suitable length, for example about 24 nucleotides, about 27 nucleotides, about 30 nucleotides, about 33 nucleotides, about 36 nucleotides, about 39 nucleotides, about 42 nucleotides, about 45 nucleotides, about 48 nucleotides, about 54 nucleotides, about 60 nucleotides, about 75 nucleotides, or about 90 nucleotides, whether or not the number of nucleic acid residues of the epitope is the same number as a string of ‘N’ designating an insertion site.

In specific, non-limiting examples, the backbone nucleic acid includes a consensus sequence having at least 90% sequence identity (such as at least 91%, at least 92%, at least 93%, at least 94%, at least 95%, at least 96%, at least 97%, at least 98%, at least 99%, or at least 100% identity) to SEQ ID NO: 3. The consensus sequence of SEQ ID NO: 3 (the part of the sequence denoted with a specific nucleotide) denotes the portion that is considered the backbone, whereas ‘N’ can vary and may include an epitope sequence (such as a heterologous or homologous epitope).

SEQ ID NO: 4 is an exemplary consensus amino acid sequence of a backbone protein. ‘X’ (or ‘Xaa’) indicates a location for insertion or substitution with a heterologous or homologous epitope. The number of “Xs” at a particular insertion or substitution site is not determinative of the amino acid length of an epitope to be inserted. Rather, the number of amino acids (‘X’) at each epitope site will vary depending on the length of the inserted epitope. A plurality of “X” is merely provided for ease of identification of each insertion site in SEQ ID NO: 4. Thus, the inserted or substituted epitope may be any suitable length, for example about 8 amino acids, about 9 amino acids, about 10 amino acids, about 11 amino acids, about 12 amino acids, about 13 amino acids, about 14 amino acids, about 15 amino acids, about 16 amino acids, about 17 amino acids, about 18 amino acids, about 20 amino acids, about 25 amino acids, or about 30 amino acids, whether or not the number of amino acid residues of the epitope is the same number as a string of “X” designating an insertion site.

In specific, non-limiting examples, the backbone protein, includes a consensus sequence having at least 90% sequence identity (such as at least 91%, at least 92%, at least 93%, at least 94%, at least 95%, at least 96%, at least 97%, at least 98%, at least 99%, or at least 100% identity) to SEQ ID NO: 4. The consensus sequence of SEQ ID NO: 4 (the part of the sequence denoted with a specific amino acid) denotes the portion that is considered the backbone, whereas ‘X’ can vary and may include an epitope sequence (such as a heterologous or homologous epitope).

### Epitopes

In some examples, the fusion protein includes at least one heterologous Vibrio cholerae epitope and may further comprise one or more homologous Vibrio cholerae epitope. A heterologous epitope is an epitope derived from a protein source other than the source of the backbone. Thus, in some examples, the heterologous Vibrio cholerae epitope is a peptide from a protein other than FlaB. In some examples, the fusion protein further includes one or more homologous Vibrio cholerae epitope (an epitope derived from the same protein source as the backbone). In some examples, a homologous epitope is a FlaB epitope.

Example epitopes (such as homologous or heterologous epitopes) include various sizes. For example, epitope size can range from 5-30 amino acids, including about 5-10, about 8-15, about 8-20, about 10-12, about 10-15, about 10-20, or about 10-30 amino acids. In some embodiments, the epitope is at least about 5 amino acids in length, for example, at least about 7, at least about 8, at least about 9, at least about 10, at least about 11, at least about 12, at least about 13, at least about 14, at least about 15, at least about 18, at least about 20, at least about 25, or at least about 30 amino acids in length. In some examples, the epitope is about 9, 10, 11, 12, or 14 amino acids. Nucleic acids that encode the epitopes are also included and can range in size from about 15-90 nucleotides, including about 15-30, about 24-45, about 24-60, about 30-36, about 30-45, about 30-60, or about 30-90 nucleotides. In some embodiments, the nucleic acid encoding an epitope is at least about 15 nucleotides, for example, at least about 21, at least about 24, at least about 27, at least about 30, at least about 33, at least about 36, at least about 39, at least about 42, at least about 45, at least about 54, at least about 60, at least about 75, or at least about 90 nucleotides in length. In some examples, the epitope is about 27, 30, 33, 36, or 42 nucleotides long.

In some embodiments, the backbone includes one or more heterologous epitope (an epitope derived from a protein source other than the source of the backbone), for example, the backbone comprises at least 1, at least 2, at least 3, at least 4, at least 5, at least 6, at least 7, at least 8, at least 9, or more heterologous epitopes. In some embodiments, the backbone comprises about 1 to about heterologous epitopes, for example, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 12 to about 20, about 14 to about 20, about 26 to about 20, about 18 to about 20, about 2 to about 10, about 3 to about 10, about 4 to about 10, about 5 to about 10, about 6 to about 10, about 7 to about 10, about 8 to about 10, about 9 to about 10, about 3 to about 9, about 4 to about 9, about 5 to about 9, about 6 to about 9, about 7 to about 9, about 8 to about 9, about 2 to about 9, about 2 to about 7, about 2 to about 6, about 2 to about 4, about 4 to about 10, about 4 to about 8, about 4 to about 6, about 6 to about 8, about 6 to about 10, about 6 to about 12, or about 8 to about 12 heterologous epitopes.

In some embodiments, the backbone is a flagellin B protein and the heterologous epitope includes a peptide of, or is derived from, a heterologous Vibrio cholerae virulence factor. Non-limiting examples of heterologous Vibrio cholerae virulence factors include toxin coregulated pilus A (TcpA), cholera toxin (CTA), cholera toxin subunit B (CTB), LPS O-antigen mimic (LPS), sialidase (Neu), hemolysin A (HlyA), flagellin C (FlaC), and flagellin D (FlaD). In some examples, the heterologous epitope is a mimic of a Vibrio cholerae virulence factor, such as the LPS O-antigen mimic (LPS). In some examples, the epitope is a peptide conserved among multiple virulence factors, for example, a peptide conserved among FlaC and FlaD (FlaC/D; e.g. Seq ID NO: 22). Exemplary heterologous Vibrio cholerae virulence factor epitopes include SEQ ID NOs: 6, 8, 10, 14, 16, 18, 20, 22, 24, 31 and 32. In a specific, non-limiting example, the heterologous Vibrio cholerae epitope has at least 90% sequence identity, such as at least 91%, at least 92%, at least 93%, at least 94%, at least 95%, at least 96%, at least 97%, at least 98%, at least 99%, or at least 100% sequence identity to one or more of SEQ ID NOs: 6, 8, 10, 14, 16, 18, 20, 22, 24, 31, and 32. In another example, the heterologous Vibrio cholerae epitope includes or consists of one or more (such as 1, 2, 3, 4, 5, 6, 7, 8, or 9) of SEQ ID NOs: 6, 8, 10, 14, 16, 18, 20, 22, 24, 31, and 32. In some embodiments, the heterologous epitope includes a peptide of, or is derived from, a heterologous Escherichia coli or Shigella virulence factor.

In some examples, the backbone further comprises one or more homologous epitope (an epitope derived from the same protein source as the backbone), for example, the backbone can include at least 1, at least 2, at least 3, at least 4, at least 5, at least 6, at least 7, at least 8, at least 9, or at least 10 homologous epitopes. In some embodiments, the backbone comprises about 1 to about 20 homologous epitopes, for example, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 12 to about 20, about 14 to about 20, about 26 to about 20, about 18 to about 20, about 2 to about 10, about 3 to about 10, about 4 to about 10, about 5 to about 10, about 6 to about 10, about 7 to about 10, about 8 to about 10, about 9 to about 10, about 3 to about 9, about 4 to about 9, about 5 to about 9, about 6 to about 9, about 7 to about 9, about 8 to about 9, about 2 to about 9, about 2 to about 7, about 2 to about 6, about 2 to about 4, about 4 to about 10, about 4 to about 8, about 4 to about 6, about 6 to about 8, about 6 to about 10, about 6 to about 12, or about 8 to about 12 homologous epitopes.

In some examples, the backbone is FlaB and the homologous epitope includes a peptide of, or is derived from, FlaB. In a specific, non-limiting example, the homologous Vibrio cholerae epitope includes at least 90% sequence identity, such as at least 91%, at least 92%, at least 93%, at least 94%, at least 95%, at least 96%, at least 97%, at least 98%, at least 99%, or at least 100% sequence identity to SEQ ID NO: 12. In another example, the homologous Vibrio cholerae epitope includes or consists of SEQ ID NO: 12.

While exemplary heterologous and homologous epitopes are provided herein; additional epitopes of use can be identified in view of the teachings and guidance disclosed herein.

### Fusion Proteins

In some embodiments, the fusion protein includes at least one backbone protein derived from FlaB and includes one or more Vibrio cholerae epitopes (e.g., one or more heterologous or homologous epitopes), for example, at least 1, at least 2, at least 3, at least 4, at least 5, at least 6, at least 7, at least 8, at least 9, or at least 10 Vibrio cholerae epitopes per backbone protein. The fusion protein is a non-naturally occurring protein, thus, the fusion protein is not, or is other than, a naturally occurring FlaB protein.

In some embodiments, the backbone comprises about 1 to about 20 Vibrio cholerae epitopes, for example, about 1 to about 19, about 1 to about 18, about 1 to about 17, about 1 to about 16, about 1 to about 15, about 1 to about 14, about 1 to about 13, about 1 to about 12, about 1 to about 11, about 1 to about 10, about 1 to about 9, about 1 to about 8, about 1 to about 7, about 1 to about 6, about 1 to about 5, about 1 to about 4, about 1 to about 3, about 1 to about 2, about 2 to about 20, about 3 to about 20, about 4 to about 20, about 5 to about 20, about 6 to about 20, about 7 to about 20, about 8 to about 20, about 9 to about 20, about 10 to about 20, about 12 to about 20, about 14 to about 20, about 26 to about 20, about 18 to about 20, about 2 to about 10, about 3 to about 10, about 4 to about 10, about 5 to about 10, about 6 to about 10, about 7 to about 10, about 8 to about 10, about 9 to about 10, about 3 to about 9, about 4 to about 9, about 5 to about 9, about 6 to about 9, about 7 to about 9, about 8 to about 9, about 2 to about 9, about 2 to about 7, about 2 to about 6, about 2 to about 4, about 4 to about 10, about 4 to about 8, about 4 to about 6, about 6 to about 8, about 6 to about 10, about 6 to about 12, or about 8 to about 12 Vibrio cholerae epitopes (e.g., heterologous or homologous epitopes) per backbone protein.

Each epitope (such as the homologous or heterologous Vibrio cholerae epitope) is independently selected, and thus may include the same or different amino acid sequence as other epitopes included in the fusion protein, and may be heterologous or homologous relative to the backbone and/or other epitopes. The epitopes may be contiguous or non-contiguous with one another in the backbone protein. In some examples, all of the epitopes are non-contiguous with one another. In other examples, one or more of the epitopes are contiguous, while one or more other epitopes are non-contiguous with the other epitopes.

In some examples, the epitopes include a peptide from one or more Vibrio cholerae virulence factors (e.g., toxin coregulated pilus A (TcpA), cholera toxin (CTA), cholera toxin subunit B (CTB), LPS O-antigen mimic (LPS), sialidase (Neu; also referred to as NanH), hemolysin A (HlyA), and flagellin C (FlaC), flagellin D (FlaD), flagellin B subunit (FlaB)). In a specific, non-limiting example, the fusion protein comprises ten Vibrio cholerae epitopes, for example, each of SEQ ID NOs: 6, 8, 10, 12, 14, 16, 18, 20, 22, and 24; or each of SEQ ID NOs: 6, 8, 10, 12, 14, 18, 20, 22, 31, and 32. In another non-limiting example, the fusion protein includes a sequence having at least about 90% sequence identity to SEQ ID NO: 2, or includes or consists of SEQ ID NO: 2.

Also disclosed herein are fusion proteins including a backbone protein, wherein the backbone protein includes a consensus sequence having at least 90% sequence identity to SEQ ID NO: 4 and the fusion protein contains at least two Vibrio cholerae epitopes (e.g., includes at least two Vibrio cholerae epitopes homologous or heterologous to the backbone), wherein at least one of the epitopes is from a different Vibrio cholerae serogroup or biotype from at least one of the other epitopes and/or from the backbone protein. Thus, the fusion protein is not a naturally occurring protein. Exemplary serogroups include Vibrio cholerae serogroup O1, serogroup O139, and non-O1/non-O139 serogroups (e.g. O3, O37, O75, O41). Exemplary biotypes include Vibrio cholerae serogroup O1 El Tor and Vibrio cholerae serogroup O1 Classical. In a non-limiting example, at least one epitope is from Vibrio cholerae biotype O1 El Tor, and at least one epitope is from Vibrio cholerae O1 Classical, O139, or a non-O1/non-O139 serogroup. In some examples, the fusion protein includes at least three Vibrio cholerae epitopes, wherein at least two of the epitopes are from a different Vibrio cholerae serogroup or biotype than the third epitope. In a non-limiting example, at least one epitope is from Vibrio cholerae biotype O1 El Tor, at least one epitope is from Vibrio cholerae O1 Classical, and at least one epitope is from Vibrio cholerae O139 or a non-O1/non-O139 serogroup. In further examples, fusion protein includes at least four Vibrio cholerae epitopes, wherein at least three of the epitopes are from a different Vibrio cholerae serogroup or biotype from the fourth epitope. In a non-limiting example, at least one epitope is from Vibrio cholerae biotype O1 El Tor, at least one epitope is from Vibrio cholerae O1 Classical, at least one epitope is from Vibrio cholerae O139, and at least one epitope is from a non-O1/non-O139 serogroup. In a specific, non-limiting example, the fusion protein includes a sequence having at least 90% sequence identity, such as at least 91%, at least 92%, at least 93%, at least 94%, at least 95%, at least 96%, at least 97%, at least 98%, at least 99%, or at least 100% sequence identity, to SEQ ID NO: 2; or the fusion protein includes or consists of SEQ ID NO: 2.

In some examples, the disclosed fusion proteins further include a tag (e.g. for purification), such as a histidine (His), chitin-binding protein (CBP), maltose-binding protein (MBP), glutathione-S-transferase (GST), or a streptavidin tag, or marker for expression of the fusion protein (e.g. a fluorescent tag such as GFP or RFP, luciferase, β-glucuronidase, β-galactosidase).

Proteins (e.g., a disclosed antigen, fusion protein, backbone protein, or epitope) and nucleic acids that are similar to those disclosed herein can be used as well as fragments thereof that retain biological activity. These proteins and nucleic acids may contain variations, substitutions, deletions, or additions. In some examples, the differences can be in regions not significantly conserved among different species. Such regions can be identified by aligning the amino acid sequences of related proteins and nucleic acids from various species. Generally, the biological effects of a molecule are retained, for example, immunogenicity or ability to elicit an immune response in a subject. For example, a protein and/or nucleic acid at least 75%, at least 80%, at least 85%, at least 90%, at least 95%, at least 96%, at least 97%, at least 98% or at least 99% identical to one of these molecules can be utilized. In some examples, proteins (or nucleic acids encoding such proteins) may include about 1, about 2, about 3, about 4, about 5, about 6, about 7, about 8, about 9, or about 10 conservative amino acid substitutions. In some examples, a protein contains no more than about 1, about 2, about 3, about 4, about 5, about 6, about 7, about 8, about 9, or about 10 conservative amino acid substitutions. Generally, modified proteins (or nucleic acids encoding such proteins) retain at least about 75%, at least about 80%, at least about 85%, at least about 90%, at least about 95%, at least about 96%, at least about 97%, at least about 98% or at least about 99% of the biological function of the native molecule or have increased biological function (such as immunogenicity) as compared to the native molecule.

Also included are derivatives or modifications of the disclosed proteins (e.g., backbone protein (such as FlaB), epitopes (such as heterologous or homologous epitopes), or fusion proteins), which are differentially modified during or after synthesis, such as by benzylation, glycosylation, acetylation, phosphorylation, amidation, pegylation, and/or derivatization by known protecting/blocking groups. In some embodiments, proteins can include at least one amino acid or every amino acid that is a D stereoisomer. Other proteins can include at least one amino acid that is reversed. The amino acid that is reversed may be a D stereoisomer. Every amino acid of a protein may be reversed and/or every amino acid can be a D stereoisomer.

Other fusion proteins can be utilized in combination with the fusion proteins disclosed herein, such as a fusion protein including enterotoxigenic Escherichia coli (ETEC) antigens or toxoid antigens (for example, as disclosed in Int. Pub. No. WO 2015/095335 A1 and Zhang et al., PLoS One 10(3):e0121623, 2015), and/or Shigella antigens (for example, as disclosed in PCT/US2020/055558). Exemplary sequences of additional fusion proteins including ETEC antigens, toxoid antigens, or Shigella antigens include SEQ ID NOs: 26, 28, and 30.

Any of the disclosed backbone proteins, epitopes, or fusion proteins can be readily synthesized by automated solid phase procedures. Techniques and procedures for solid phase synthesis are described, for example, in Solid Phase Peptide Synthesis: A Practical Approach, by E. Atherton and R. C. Sheppard, published by IRL, Oxford University Press, 1989. Alternatively, these proteins, epitopes, or fusion proteins may be prepared by way of segment condensation, as described, for example, in Liu et al., Tetrahedron Lett. 37:933-936, 1996; Baca et al., J. Am. Chem. Soc. 117:1881-1887, 1995; Tam et al., Int. J. Peptide Protein Res. 45:209-216, 1995; Schnolzer and Kent, Science 256:221-225, 1992; Liu and Tam, J. Am. Chem. Soc. 116:4149-4153, 1994; Liu and Tam, Proc. Natl. Acad. Sci. USA 91:6584-6588, 1994; and Yamashiro and Li, Int. J. Peptide Protein Res. 31:322-334, 1988). Other methods useful for synthesizing peptides of the present disclosure are described in Nakagawa et al., J. Am. Chem. Soc. 107:7087-7092, 1985.

Any of the disclosed backbone proteins, epitopes, or fusion proteins can also be obtained through cell-based expression systems, such as expressing respective nucleic acid sequences in BL21 (DE3) competent Escherichia coli, yeast (such as Saccharomyces cerevisiae or Kluyveromyces lactis), Spodoptera frugiperda Sf9 cells, Chinese hamster ovary (CHO) cells, baby hamster kidney (BHK21) cells, human embryonic kidney (HEK 293), or murine myeloma cells (NS0 and Sp2/0).

### II. Nucleic Acids, Vectors, and Host Cells

Also disclosed herein are nucleic acids encoding the disclosed proteins (e.g., fusion protein, backbone protein, antigens), and vectors encoding such nucleic acids. In some embodiments, the nucleic acid encodes an amino acid consensus sequence (backbone sequence) having at least 90% sequence identity (such as at least 91%, at least 92%, at least 93%, at least 94%, at least 95%, at least 96%, at least 97%, at least 98%, at least 99%, or at least 100% identity) to SEQ ID NO: 4. In some examples, the nucleic acid encodes an amino acid consensus sequence that includes or consists of SEQ ID NO: 4. In some examples, the nucleic acid further encodes at least one heterologous epitope, for example, the nucleic acid encodes one or more of (such as 1, 2, 3, 4, 5, 6, 7, 8, 9, or 10) SEQ ID NOs: 6, 8, 10, 14, 16, 18, 20, 22, 24, 31, and 32. In some examples, the nucleic acid encodes at least one homologous epitope, for example, the nucleic acid encodes SEQ ID NO: 12. In some examples, the nucleic acid encodes an amino acid sequence having at least 90% sequence identity (such as at least 91%, at least 92%, at least 93%, at least 94%, at least 95%, at least 96%, at least 97%, at least 98%, at least 99%, or at least 100% identity) to each of SEQ ID NOs: 6, 8, 10, 12, 14, 16, 18, 20, 22, and 24, respectively. In further examples, the nucleic acid encodes each of SEQ ID NOs: 6, 8, 10, 12, 14, 16, 18, 20, 22, and 24. In some examples, the nucleic acid encodes an amino acid sequence having at least 90% sequence identity (such as at least 91%, at least 92%, at least 93%, at least 94%, at least 95%, at least 96%, at least 97%, at least 98%, at least 99%, or at least 100% identity) to each of SEQ ID NOs: 6, 8, 10, 12, 14, 18, 20, 22, 31, and 32, respectively. In further examples, the nucleic acid encodes each of SEQ ID NOs: 6, 8, 10, 12, 14, 18, 20, 22, 31, and 32.

In some embodiments, the nucleic acid includes a consensus sequence (backbone sequence) having at least 90% sequence identity (such as at least 91%, at least 92%, at least 93%, at least 94%, at least 95%, at least 96%, at least 97%, at least 98%, at least 99%, or at least 100% identity) to SEQ ID NO: 3. In some examples, the nucleic acid includes a consensus sequence that includes or consists of SEQ ID NO: 3. In some examples, the nucleic acid further encodes at least one heterologous epitope, for example, the nucleic acid includes one or more of (such as 1, 2, 3, 4, 5, 6, 7, 8, or all of) SEQ ID NOs: 5, 7, 9, 13, 15, 17, 19, 21, and 23. In some examples, the nucleic acid encodes at least one homologous epitope, for example, the nucleic acid includes SEQ ID NO: 11. In some examples, the nucleic acid includes nucleic acid sequences having at least 90% sequence identity (such as at least 91%, at least 92%, at least 93%, at least 94%, at least 95%, at least 96%, at least 97%, at least 98%, at least 99%, or at least 100% identity) to each of SEQ ID NOs: 5, 7, 9, 11, 13, 15, 17, 19, 21, and 23, respectively. In further examples, the nucleic acid includes each of SEQ ID NOs: 5, 7, 9, 11, 13, 15, 17, 19, 21, and 23.

In some examples, the nucleic acid encodes a consensus sequence having at least 90% sequence identity (such as at least 91%, at least 92%, at least 93%, at least 94%, at least 95%, at least 96%, at least 97%, at least 98%, at least 99%, or at least 100% identity) to SEQ ID NO: 3, and encodes at least two Vibrio cholerae epitopes (independently homologous or heterologous to the backbone), wherein at least one of the epitopes is from a different Vibrio cholerae serogroup or biotype. Exemplary serogroups include O1, O139, and non-O1/O139 (e.g. O3, O37, O75, O141). Exemplary biotypes include O1 El Tor and O1 Classical. In some examples, the nucleic acid encodes at least three Vibrio cholerae epitopes (independent homologous or heterologous to the backbone), wherein at least two of the epitopes are from a different Vibrio cholerae serogroup or biotype from the third epitope. In some examples, the nucleic acid encodes at least four Vibrio cholerae epitopes (independently homologous or heterologous to the backbone), wherein at least three of the epitopes are from a different Vibrio cholerae serogroup or biotype than the fourth epitope.

In some examples, the nucleic acid sequence encodes an amino acid sequence having at least 90% sequence identity (such as at least 91%, at least 92%, at least 93%, at least 94%, at least 95%, at least 96%, at least 97%, at least 98%, at least 99%, or at least 100% identity) to SEQ ID NO: 2. In some examples, the nucleic acid encodes an amino acid sequence including or consisting of SEQ ID NO: 2. In some examples, the nucleic acid sequence includes a sequence having at least 90% sequence identity (such as at least 91%, at least 92%, at least 93%, at least 94%, at least 95%, at least 96%, at least 97%, at least 98%, at least 99%, or at least 100% identity) to SEQ ID NO: 1.

In further examples, the nucleic acid includes or consists of SEQ ID NO: 1.

In some examples, the disclosed nucleic acids further encode a tag for purification of the fusion protein (e.g., histidine (His), chitin-binding protein (CBP), maltose-binding protein (MBP), or glutathione-S-transferase (GST), or a streptavidin tag), or marker for expression of the fusion protein (e.g. fluorescent tag such as GFP or RFP, luciferase, β-glucuronidase, β-galactosidase).

The disclosed nucleic acids include DNA, cDNA and RNA sequences which encode the fusion protein, for example, including the nucleic acid sequences disclosed herein. The coding sequence includes variants that result from the degeneracy (e.g., redundancy) of the genetic code, whereby more than one codon can encode the same amino acid residue. Thus, for example, leucine can be encoded by CTT, CTC, CTA, CTG, TTA, or TTG; serine can be encoded by TCT, TCC, TCA, TCG, AGT, or AGC; asparagine can be encoded by AAT or AAC; aspartic acid can be encoded by GAT or GAC; cysteine can be encoded by TGT or TGC; alanine can be encoded by GCT, GCC, GCA, or GCG; glutamine can be encoded by CAA or CAG; tyrosine can be encoded by TAT or TAC; and isoleucine can be encoded by ATT, ATC, or ATA. Tables showing the standard genetic code can be found in various sources (see e.g., Stryer, 1988, Biochemistry, 3rd Edition, W.H. 5 Freeman and Co., NY).

In addition, the disclosed nucleic acids may be codon-optimized for expression in a given organism. Codon usage bias, the use of synonymous codons at unequal frequencies, is ubiquitous among genetic systems (Ikemura, J. Mol. Biol. 146:1-21, 1981; Ikemura, J. Mol. Biol. 158:573-97, 1982). The strength and direction of codon usage bias is related to genomic G+C content and the relative abundance of different isoaccepting tRNAs (Akashi, Curr. Opin. Genet. Dev. 11:660-666, 2001; Duret, Curr. Opin. Genet. Dev. 12:640-9, 2002; Osawa et al., Microbiol. Rev. 56:229-264, 1992). Codon usage can affect the efficiency of gene expression. Codon-optimization refers to replacement of at least one codon (such as at least 5 codons, at least 10 codons, at least 25 codons, at least 50 codons, at least 75 codons, at least 100 codons or more) in a nucleic acid sequence with a synonymous codon (one that codes for the same amino acid) more frequently used (preferred) in the organism. Each organism has a particular codon usage bias for each amino acid, which can be determined from publicly available codon usage tables (for example see Nakamura et al., Nucleic Acids Res. 28:292, 2000 and references cited therein). For example, a codon usage database is available on the World Wide Web at kazusa.or.jp/codon. One of skill in the art can modify a nucleic acid encoding a particular amino acid sequence, such that it encodes the same amino acid sequence, while being optimized for expression in a particular cell type. In some examples, the disclosed fusion protein is codon optimized for expression in a particular organism, for example, Escherichia coli, yeast (such as Saccharomyces cerevisiae or Kluyveromyces lactis), Spodoptera frugiperda, mouse, hamster, or human.

A nucleic acid encoding the fusion protein can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a nucleic acid encoding the protein can be isolated by polymerase chain reaction of cDNA using primers based on the DNA sequence of the molecule. A wide variety of cloning and in vitro amplification methodologies are well-known to persons skilled in the art. PCR methods are described in, for example, U.S. Pat. No. 4,683,195; Mullis et al., Cold Spring Harbor Symp. Quant. Biol. 51:263, 1987; and Erlich, ed., PCR Technology, (Stockton Press, N Y, 1989). Nucleic acids can also be isolated by screening genomic or cDNA libraries with probes selected from the sequences of the desired nucleic acids under hybridization conditions.

A nucleic acid encoding the fusion protein can be operably linked to one or more expression control sequences. An expression control sequence operably linked to a coding sequence is linked such that expression of the coding sequence is achieved under conditions compatible with the expression control sequences. The expression control sequences include, but are not limited to, appropriate promoters, enhancers, ribosomal binding sites, transcription terminators, transcriptional regulators (e.g., AraC and LacI), start codons (e.g., ATG) in front of a protein-encoding gene, splicing signal for introns, insertions to maintain the correct reading frame of the gene and permit proper translation of mRNA, and/or stop codons.

The nucleic acid encoding the fusion protein includes recombinant DNA, and can be incorporated into a vector, such as a plasmid or viral vector, or into the genomic DNA of a prokaryote or eukaryote, or may exist as a separate molecule (such as a cDNA) independent of other sequences. The nucleotides can be ribonucleotides, deoxyribonucleotides, or modified forms of either nucleotide. The term includes single and double stranded forms of DNA.

In some examples, a vector includes a nucleic acid encoding at least one fusion protein disclosed herein. A person of skill in the art will understand that there are many options for vectors, for example, plasmid, viral vector, cosmid, or artificial chromosome (e.g. bacterial, yeast, human), and could readily identify a suitable vector for a desired application. In some examples, the vector is pET28a. pET28a is suitable for expression in bacteria, for example, expression in E. coli BL21 (DE3). A number of viral vectors have been constructed, including polyoma, SV40 (Madzak et al., J. Gen. Virol., 73:15331536, 1992), adenovirus (Berkner, Cur. Top. Microbiol. Immunol., 158:39-6, 1992; Berliner et al., Bio Techniques, 6:616-629, 1998; Gorziglia et al., J. Virol., 66:4407-4412, 1992; Quantin et al., Proc. Natl. Acad. Sci. USA, 89:2581-2584, 1992; Rosenfeld et al., Cell, 68:143-155, 1992; Wilkinson et al., Nucl. Acids Res., 20:2233-2239, 1992; Stratford-Perricaudet et al., Hum. Gene Ther., 1:241-256, 1990), vaccinia virus (Mackett et al., Biotechnology, 24:495-499, 1992), adeno-associated virus (Muzyczka, Curr. Top. Microbiol. Immunol., 158:91-123, 1992; On et al., Gene, 89:279-282, 1990), herpes viruses including HSV and EBV (Margolskee, Curr. Top. Microbiol. Immunol., 158:67-90, 1992; Johnson et al., J. Virol., 66:29522965, 1992; Fink et al., Hum. Gene Ther. 3:11-19, 1992; Breakfield et al., Mol. Neurobiol., 1:337-371, 1978; Fresse et al., Biochem. Pharmacol., 40:2189-2199, 1990), Sindbis viruses (H. Herweijer et al., Human Gene Therapy, 6:1161-1167, 1995; U.S. Pat. Nos. 5,091,309 and 5,2217,879), alphaviruses (S. Schlesinger, Trends Biotechnol. 11:18-22, 1993; I. Frolov et al., Proc. Natl. Acad. Sci. USA, 93:11371-11377, 1996) and retroviruses of avian (Brandyopadhyay et al., Mol. Cell Biol., 4:749-754, 1984; Petropouplos et al., J. Virol., 66:3391-3397, 1992), murine (Miller, Curr. Top. Microbiol. Immunol., 158:1-24, 1992; Miller et al., Mol. Cell Biol., 5:431-437, 1985; Sorge et al., Mol. Cell Biol., 4:1730-1737, 1984; Mann et al., J. Virol., 54:401-407, 1985), and human origin (Page et al., J. Virol., 64:5370-5276, 1990; Buchschalcher et al., J. Virol., 66:2731-2739, 1992). Baculovirus (Autographa californica multinuclear polyhedrosis virus; AcMNPV) vectors are also known in the art, and may be obtained from commercial sources (such as PharMingen, San Diego, Calif.; Protein Sciences Corp., Meriden, Conn.; Stratagene, La Jolla, Calif.). In some examples, the vector is a viral vector (e.g., retrovirus vectors, orthopox vectors, avipox vectors, fowlpox vectors, capripox vectors, suipox vectors, adenoviral vectors, adeno-associated viral vectors, herpes virus vectors, alpha virus vectors, baculovirus vectors, Sindbis virus vectors, vaccinia virus vectors, and poliovirus vectors).

Nucleic acid encoding the fusion protein can be expressed in vitro by transfer of the nucleic acid into a suitable host cell. The cell may be prokaryotic or eukaryotic. The term also includes any progeny of the subject host cell. It is understood that all progeny may not be identical to the parental cell since there may be mutations that occur during replication. Methods of stable transfer, meaning that the foreign DNA is continuously maintained in the host, are known in the art.

Hosts cells also can include microbial, insect, and mammalian host cells. Methods of expressing nucleic acids in host cells are well known in the art. Non-limiting examples of suitable host cells include bacteria, archaea, insect, fungi (for example, yeast), plant, and animal cells (for example, mammalian cells, such as human). Exemplary cells of use include Escherichia coli, Bacillus subtilis, Saccharomyces cerevisiae, Salmonella typhimurium, yeast (such as Saccharomyces cerevisiae or Kluyveromyces lactis), Spodoptera frugiperda (SF9 cells), C129 cells, 293 cells, Neurospora, and immortalized mammalian myeloid and lymphoid cell lines. Examples of commonly used mammalian host cell lines include VERO, HeLa cells, human embryonic kidney cells (HEK 293), CHO cells, baby hamster kidney cells (e.g., BHK21), mouse NS0 or Sp2/0 myeloma cells, WI38, BHK, and COS cell lines, although other cell lines may be used, such cells are designed to provide higher expression, desirable glycosylation patterns, or other features.

Transformation or transfection of a host cell with the disclosed nucleic acids or vectors encoding the disclosed nucleic acids, can be carried out through a variety of techniques. Where the host is prokaryotic, such as, but not limited to, Escherichia coli competent cells, which are capable of DNA uptake, can be prepared from cells harvested after exponential growth phase and subsequently treated by the CaCl₂ method (chemical transformation). Alternatively, MgCl₂ or RbCl can be used. Transformation can also be performed after forming a protoplast of the host cell if desired, or by electroporation or particle bombardment.

When the host is a eukaryote, such methods of transfection of DNA include calcium phosphate coprecipitates, mechanical procedures (such as microinjection), electroporation, insertion of a plasmid encased in liposomes, or viral vectors can be used. Eukaryotic cells can also be co-transformed with polynucleotide sequences encoding a polypeptide, and a second foreign DNA molecule encoding a selectable phenotype, such as the herpes simplex thymidine kinase gene. Another method is to use a eukaryotic viral vector, such as one of the viral vectors described herein, to transiently infect or transform eukaryotic cells and express the protein.

In some examples, the disclosed nucleic acids are expressed in a cell-based protein expression system. A person of skill in the art will understand that there are many options for protein expression systems, including systems that express protein in yeast, insect, bacterial, or mammalian cells. Non-limiting examples of cell lines suitable for expressing the disclosed nucleic acids or vectors comprising the nucleic acids include BL21 (DE3) competent E. coli, yeast (such as Saccharomyces cerevisiae or Kluyveromyces lactis), Spodoptera frugiperda S19 cells, Chinese hamster ovary (CHO) cells, baby hamster kidney (BHK21) cells, human embryonic kidney (HEK 293), and murine myeloma cells (NS0 and Sp2/0). In some examples, the cells are transformed or transfected with an expression vector including the disclosed nucleic acids. The expression vector can also include one or more tags for purification of the fusion protein, such as histidine (His), chitin-binding protein (CBP), maltose-binding protein (MBP), or glutathione-S-transferase (GST), or a streptavidin tag. In specific, non-limiting examples, the vector contains a His tag, such as a pET28a vector.

In some examples, the disclosed nucleic acids are expressed in yeast, such as Saccharomyces cerevisiae or Kluyveromyces lactis. In some examples, the yeast includes an expression vector comprising the disclosed nucleic acids. Several promoters are known to be of use in yeast expression systems such as the constitutive promoters plasma membrane H⁺-ATPase (PMA1), glyceraldehyde-3-phosphate dehydrogenase (GPD), phosphoglycerate kinase-1 (PGK1), alcohol dehydrogenase-1 (ADH1), and pleiotropic drug-resistant pump (PDR5). In addition, many inducible promoters are of use, such as GAL1-10 (induced by galactose), PHOS (induced by low extracellular inorganic phosphate), and tandem heat shock HSE elements (induced by temperature elevation to 37° C.). Promoters that direct variable expression in response to a titratable inducer include the methionine-responsive MET3 and MET25 promoters and copper-dependent CUP1 promoters. Any of these promoters may be cloned into multicopy (2p) or single copy (CEN) plasmids to give an additional level of control in expression level. The plasmids can include nutritional markers (such as URA3, ADE3, HIS1, and others) for selection in yeast and antibiotic resistance (such as AMP) for propagation in bacteria. Plasmids for expression on K lactis are known, such as pKLAC1. Thus, in one example, after amplification in bacteria, plasmids can be introduced into the corresponding yeast auxotrophs by methods similar to bacterial transformation. The polynucleotides can also be designed to express in insect cells (such as Spodoptera).

The fusion protein can be expressed in a variety of yeast strains. For example, seven pleiotropic drug-resistant transporters, YOR1, SNQ2, PDR5, YCF1, PDR10, PDR11, and PDR15, together with their activating transcription factors, PDR1 and PDR3, have been simultaneously deleted in yeast host cells, rendering the resultant strain sensitive to drugs. Yeast strains with altered lipid composition of the plasma membrane, such as the erg6 mutant defective in ergosterol biosynthesis, can also be utilized. Proteins that are highly sensitive to proteolysis can be expressed in a yeast lacking the master vacuolar endopeptidase Pep4, which controls the activation of other vacuolar hydrolases. Heterologous expression in strains carrying temperature-sensitive (ts) alleles of genes can be employed if the corresponding null mutant is inviable.

While molecular methods can be used to synthesize the disclosed fusion proteins, chemical methods can alternatively be used to link a peptide (such as an epitope) to a backbone protein. In some examples, peptides from a backbone protein (such as FlaB) can be covalently bound to epitope(s) (such as epitopes from TcpA, CT, CTB, LPS mimic, Neu, HlyA, FlaC, FlaD, and FlaB) through chemical conjugation. Various types of chemical reagents can be used (see, e.g., Ido et al., JBC, 287(31): 26377-26387, 2012, and U.S. Pat. Pub. No. 2003/0040496, both incorporated herein by reference). In some examples, 1-ethyl-3-(3-dimethylaminopropyl)carbodiimide hydrochloride (EDC) and/or N-hydroxysulfosuccinimide (Sulfo-NHS) is used for chemical conjugation (see, e.g., Ido et al., JBC, 287(31): 26377-26387, 2012, incorporated herein by reference) In other embodiments, alternative chemical conjugation (e.g., cross-linking) reagents may be used to form covalent bonds between amino groups and thiol groups and to introduce thiol groups into proteins (see, e.g., U.S. Pat. Pub. No. 2003/0040496, incorporated herein by reference). Additional alternative chemical conjugation (e.g., cross-linking) reagents can be found in the PIERCE CATALOG, ImmunoTechnology Catalog & Handbook, 1992-1993, which describes the preparation of and use of such reagents and provides a commercial source for such reagents (incorporated herein by reference; see also, e.g., Cumber et al., Bioconjugate Chem. 3:397-401, 1992; Thorpe et al., Cancer Res. 47:5924-5931, 1987; Gordon et al., Proc. Natl. Acad Sci. 84:308-312, 1987; Walden et al., J. Mol. Cell Immunol. 2:191-197, 1986; Carlsson et al., Biochem. J. 173:723-737, 1978; Mahan et al., Anal. Biochem., 162:163-170, 1987; Wawryznaczak et al., Br. J. Cancer 66:361-366, 1992; Fattom et al., Infection & Immun. 60:584-589, 1992). These reagents include, but are not limited to: N-succinimidyl-3-(2-pyridyldithio)propionate (SPDP; disulfide linker); sulfosuccinimidyl 6-[3-(2-pyridyldithio)propionamido]hexanoate (sulfo-LC-SPDP); succinimidyloxycarbonyl-α-methyl benzyl thiosulfate (SMBT, hindered disulfate linker); succinimidyl 6-[3-(2-pyridyldithio) propionamido]hexanoate (LC-SPDP); sulfosuccinimidyl 4-(N-maleimidomethyl)cyclohexane-1-carboxylate (sulfo-SMCC); succinimidyl 3-(2-pyridyldithio)butyrate (SPDB; hindered disulfide bond linker); sulfosuccinimidyl 2-(7-azido-4-methylcoumarin-3-acetamide) ethyl-1,3′-dithiopropionate (SAED); sulfosuccinimidyl 7-azido-4-methylcoumarin-3-acetate (SAMCA); sulfosuccinimidyl 6-[alpha-methyl-alpha-(2-pyridyldithio)toluamido]hexanoate (sulfo-LC-SMPT); 1,4-di-[3′-(2′-pyridyldithio)propionamido]butane (DPDPB); 4-succinimidyloxycarbonyl-methyl-(2-pyridylthio)toluene (SMPT, hindered disulfate linker); sulfosuccinimidyl6[-methyl-2-pyridyldithio)toluamido]hexanoate (sulfo-LC-SMPT); m-maleimidobenzoyl-N-hydroxysuccinimide ester (MBS); m-maleimidobenzoyl-N-hydroxysulfosuccinimide ester (sulfo-MBS); N-succinimidyl(4-iodoacetyl)aminobenzoate (SIAB; thioether linker); sulfosuccinimidyl(4-iodoacetyl)amino benzoate (sulfo-SIAB); succinimidyl4(p-maleimidophenyl)butyrate (SMPB); sulfosuccinimidyl-4-(p-maleimidophenyl)butyrate (sulfo-SMPB); and azidobenzoyl hydrazide (ABH).

### III. Pharmaceutical and Immunogenic Compositions

Pharmaceutical compositions provided herein include the disclosed fusion protein, and nucleic acids and vectors encoding the fusion protein, and a pharmaceutically acceptable carrier. Also provided herein are immunogenic compositions comprising the fusion protein, and nucleic acids and vectors encoding the fusion protein, or the pharmaceutical composition disclosed herein. The pharmaceutical compositions and immunogenic compositions disclosed herein are collectively referred to as “compositions.” Such compositions can be administered to subjects by a variety of administration modes known to the person of ordinary skill in the art, for example, intramuscular, subcutaneous, intravenous, intra-arterial, intra-articular, intraperitoneal, intradermal, or parenteral routes. In specific examples, the compositions can be administered via subcutaneous, intradermal, or intramuscular routes. In a specific non-limiting example, the compositions are administered via an intramuscular route. Actual methods for preparing administrable compositions are described in more detail in publications such as Remington's Pharmaceutical Sciences, by E.W. Martin, Mack Publishing Co., Easton, PA, 22nd Edition, 2013.

The composition can be formulated with pharmaceutically acceptable carriers to help retain biological activity while also promoting desirable characteristics such as increased stability during storage within an acceptable temperature range. Potential carriers include, but are not limited to, physiologically balanced solutions, phosphate buffered saline, water, emulsions (for example, oil/water or water/oil emulsions), various types of wetting agents, cryoprotective additives or stabilizers such as proteins, peptides or hydrolysates (for example, albumin, gelatin), sugars (for example, sucrose, lactose, sorbitol), amino acids (for example, sodium glutamate), or other protective agents. The resulting aqueous solutions may be packaged for use as is or lyophilized. Lyophilized preparations are combined with a sterile solution prior to administration for either single or multiple dosing.

Formulated compositions, especially liquid formulations, may contain a bacteriostat to prevent or minimize degradation during storage, including but not limited to effective concentrations (usually <1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

The compositions of the disclosure can contain, as pharmaceutically acceptable carriers, substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and/or triethanolamine oleate.

The composition may optionally include an adjuvant to enhance an immune response of a subject. Adjuvants, such as aluminum hydroxide (ALHYDROGEL®, available from Brenntag Biosector, Copenhagen, Denmark and AMPHOGEL®, Wyeth Laboratories, Madison, NJ), Freund's adjuvant, MPL™ (3-O-deacylated monophosphoryl lipid A; Corixa, Hamilton, IN), IL-12 (Genetics Institute, Cambridge, MA), TLR agonists (such as TLR-9 agonists), among many other suitable adjuvants, can be included in the compositions. Suitable adjuvants include, for example, toll-like receptor agonists, alum, AlPO₄, alhydrogel, Lipid-A and derivatives or variants thereof, dmLT, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants help to stimulate the immune system in a non-specific way, thus enhancing the immune response to a pharmaceutical product. In some embodiments, the adjuvant is selected to elicit a Th1 biased immune response in a subject.

In some examples, the adjuvant formulation includes a mineral salt, such as a calcium or aluminum (alum) salt, for example calcium phosphate, aluminum phosphate or aluminum hydroxide. In some embodiments, the adjuvant includes an oil and water emulsion, for example, an oil-in-water emulsion (such as MF59 (Novartis) or AS03 (GlaxoSmithKline). One example of an oil-in-water emulsion comprises a metabolizable oil, such as squalene, a tocol such as a tocopherol, for example, alpha-tocopherol, and a surfactant, such as sorbitan trioleate (Span 85) or polyoxyethylene sorbitan monooleate (Tween 80), in an aqueous carrier. In other examples, the adjuvant formulation includes dmLT.

In some instances, it may be desirable to combine a disclosed composition with other pharmaceutical products (for example, other vaccines, such as Ty21a, or other fusion proteins, such as those including ETEC antigens, ETEC toxoid antigens, or Shigella antigens, for example, ETEC antigens CFA/I/II/IV MEFA (e.g. SEQ ID NO: 26), and/or toxoid fusion 3xSTa_{N12S}-mnLT_{R192G/L211A}, (e.g. SEQ ID NO: 28), or Shigella MEFA (e.g. SEQ ID NO: 30)), which induce immune responses to other agents. For example, a composition including a fusion protein or nucleic acid as described herein can be administered simultaneously or sequentially with other vaccines, for example, vaccines recommended by the Advisory Committee on Immunization Practices (ACIP; cdc.gov/vaccines/acip/index) for the targeted age group (for example, children at or less than 5 years old, children at or less than three years old, children at or less than one year old, children over 5 years old, children over 12 years old, or adults). As such, a disclosed composition described herein may be administered simultaneously or sequentially with vaccines against, for example, ETEC, typhoid (for example, Ty21a), Shigella (e.g. shigellosis), measles virus, rubella virus, varicella zoster virus, hepatitis B (HepB), diphtheria, tetanus and pertussis (DTaP), pneumococcal bacteria (PCV), Haemophilus influenzae type b (Hib), polio, influenza, and/or rotavirus. In some examples, the composition further comprises at least one additional fusion protein. In some examples, the additional fusion protein comprises enterotoxigenic Escherichia coli and/or toxoid antigens. In specific, non-limiting examples, the additional fusion protein includes or consists of SEQ ID NO: 26 or 28.

In some embodiments, the composition can be provided as a sterile composition. The composition typically contains an effective amount of a disclosed composition. Typically, the amount of the composition in each dose is selected as an amount that induces an immune response without significant, adverse side effects. In some embodiments, the composition can be provided in unit dosage form for use to induce an immune response in a subject, for example, an amount that prevents or inhibits Vibrio cholerae infection in the subject. A unit dosage form contains a suitable single preselected dosage for administration to a subject, suitable marked or measured multiples of two or more preselected unit dosages, and/or a metering mechanism for administering the unit dose or multiples thereof.

Also provided herein are live attenuated bacterial vaccines including the disclosed nucleic acids or vectors encoding the disclosed fusion protein in an attenuated pathogen. In some examples, the attenuated pathogen is a vaccine strain of Vibrio cholerae, Shigella flexneri, Salmonella enterica, Salmonella typhi, Yersinia enterocolitica, Listeria monocytogenes, Bordetella bronhiseptica, Erysipelotrix rhusiopatie, Mycobacterium bovis, or Brucella abortus. In some examples, the attenuated pathogen is Salmonella typhi Ty21a. In some examples, the attenuated pathogen is used as a vehicle to deliver the disclosed nucleic acids or vectors to a subject. Methods of using attenuated pathogens as a vaccine vehicle are described, for example, by Detmer and Glenting (2006) Microb Cell Fact. 5:23.

The live attenuated bacterial vaccine can be formulated with pharmaceutically acceptable carriers to help retain biological activity and/or promote increased stability during storage, a bacteriostat to prevent or minimize degradation during storage, or adjuvant, as described herein. The live attenuated bacterial vaccine can be administered to a subject by a variety of administration modes, for example, oral, intranasal, intramuscular, subcutaneous, intravenous, intra-arterial, intra-articular, intraperitoneal, intradermal, or parenteral routes. In some examples, the vaccine is administered via oral or intranasal routes. In a specific non-limiting example, administration of the live attenuated bacterial vaccine is oral. The amount of the live attenuated bacterial vaccine in each dose is selected as an amount that induces an immune response without significant, adverse side effects. In some embodiments, the vaccine can be provided in unit dosage form for use to induce an immune response in a subject, for example, to prevent or inhibit Vibrio cholerae infection in the subject. A unit dosage form contains a suitable single preselected dosage for administration to a subject, suitable marked or measured multiples of two or more preselected unit dosages, and/or a metering mechanism for administering the unit dose or multiples thereof.

### IV. Method of Inducing an Immune Response

In some embodiments, methods of inducing an immune response to Vibrio cholerae in a subject are provided. The disclosed methods include administering one or more of the disclosed compositions (e.g., the disclosed fusion protein, the nucleic acid or vector encoding the fusion protein, the pharmaceutical composition, the immunogenic composition, or the live attenuated bacterial vaccine) to a subject. In some embodiments, the subject is a human. In some examples, the methods induce an immune response to Vibrio cholerae (such as one or more of Vibrio cholerae O1, O139, or a non-O1/O139 serogroup) in the subject. In some examples, at least one additional fusion protein is included in the composition, such as an additional fusion protein with enterotoxigenic Escherichia coli antigens, toxoid antigens, or Shigella antigens. Is such examples, the method can also induce an immune response to Escherichia coli (such as enterotoxigenic Escherichia coli) or Shigella in the subject. The immune response can be a protective immune response, for example, a response that inhibits or reduces subsequent infection by Vibrio cholerae. Eliciting the immune response can also be used to treat or inhibit Vibrio cholerae infection and illnesses associated therewith.

Typical subjects for administration or treatment with the disclosed compositions and methods of the present disclosure include humans and any other animals susceptible to infection by Vibrio cholerae. In some examples, a subject is selected for treatment that has, or is at risk for developing cholera, for example, due to exposure or the possibility of exposure to Vibrio cholerae. In some examples, a subject is selected that is in or traveling to an area with endemic Vibrio cholerae.

In some examples, the composition is administered to an adult, for example, an adult over the age of 18, over the age of 25, over the age of 35, over the age of 45, over the age of 55, or over the age of 65, over the age of 70, over the age of 75, or over the age of 80. In some examples, the adult is about 18 to 30 years old, about 30 to 55 years of age, about 30 to 65 years old, about 18 to years old, about 18 to 55 years old, about 18 to 65 years old, about 18 to 70 years old, about 18 to 80 years old, or about 18 to 90 years.

In some examples, the composition is administered to child, for example, a child under the age of 18, under the age of 16, under the age of 12, under the age of 8, under the age of 6, under the age of 5, under the age of 4, under the age of 3, under the age of 2, or under the age of 1. In some examples, the child is about 3 months to about 12 months old, about 6 months to about 18 months, about 1 to about 3 years old, about newborn to about 5 years old, about 3 months to about 5 years old, about 6 months to about 5 years old, about 9 months to about 5 years old, about 1 to about 5 years old, about 3 to about 5 years old, about 1 to about 6 years old, about 3 to about 6, about 5 to about 8 years old, about 8 to about 12 years old, about 12 to about 16 years old, about 14 to about 18 years old, or about 16 to about 18 years old.

The disclosed compositions can be administered, for example, by beginning an immunization regimen any time from about newborn to 95 years old, for example, an immunization regimen can begin from about newborn to 12 months of age, about 3 months to 12 months of age, about 6 months to 12 months of age, about 9 months to 12 months of age, about 12 months to 15 months of age, about 2 years to 5 years of age, about 3 years to 6 years of age, about 4 years to 8 years of age, about 6 years to 12 years of age, about 10 years to 14 years of age, about 12 years to 18 years of age, about 18 years to 65 years of age, or about 18 years to 95 years of age. In some examples, the immunization regimen begins any time after about 1 year of age, about 2 years of age, about 3 years of age, about 4 years of age, about 5 years of age, about 12 years of age, about 18 years of age, or any time after about 65 years of age. In particular examples, a child is administered a first dose at 12-15 months of age and a second dose between 4-6 years of age.

Booster doses at later ages can also be administered, such as if it is determined that the subject exhibits waning immunity against Vibrio cholerae infection. In other examples, the compositions are administered to a subject prior to travel to an area where Vibrio cholerae is endemic, for example 1 to 12 months (such as 1 to 3 months, 2 to 6 months, 4 to 8 months, or 8 to 12 months) prior to travel. Booster doses can also be administered prior to travel.

Administration of the disclosed compositions (e.g., the fusion protein, nucleic acid or vector encoding the fusion protein, the pharmaceutical composition, the immunogenic composition, or the live attenuated bacterial vaccine) can be for prophylactic or therapeutic purpose. When provided prophylactically, the compositions can be provided in advance of any symptom, for example, in advance of infection. Thus, in some examples, the method comprises selecting a subject that does not have, or is not suspected of having, cholera or a Vibrio cholerae infection. The prophylactic administration serves to inhibit or ameliorate any subsequent infection. In some embodiments, the methods can involve selecting a subject at risk for contracting Vibrio cholerae infection and administering a therapeutically effective amount of a disclosed compositions to the subject.

The disclosed compositions (e.g., the fusion protein, nucleic acid or vector encoding the fusion protein, the pharmaceutical composition, the immunogenic composition, or the live attenuated bacterial vaccine) can be provided prior to anticipated exposure to Vibrio cholerae so as to attenuate the anticipated severity, duration, or extent of an infection and/or associated disease symptoms; after exposure or suspected exposure to the pathogen; or after the actual initiation of an infection. When provided therapeutically, the disclosed compositions are provided at or after the onset of a symptom of Vibrio cholerae infection (such as developing symptoms of cholera) or after diagnosis of Vibrio cholerae infection or cholera.

In some embodiments, administration of the disclosed compositions (e.g., the fusion protein, nucleic acid or vector encoding the fusion protein, the pharmaceutical composition, the immunogenic composition, or the live attenuated bacterial vaccine) can elicit the production of an immune response that is protective against or reduces the severity of symptoms or complications of Vibrio cholerae when the subject is subsequently infected or re-infected with Vibrio cholerae. While the naturally circulating pathogen may still be capable of causing infection, there can be a reduced possibility of symptoms as a result of the vaccination and a possible increase in resistance to subsequent infection by Vibrio cholerae. Following administration of a disclosed composition, the subject can be monitored for the development of antibodies to Vibrio cholerae, and/or monitored for signs or symptoms of subsequent Vibrio cholerae infection or disease (cholera).

The compositions described herein (e.g., the fusion protein, nucleic acid or vector encoding the fusion protein, the pharmaceutical composition, the immunogenic composition, or the live attenuated bacterial vaccine) are administered to a subject in an amount effective to induce or enhance an immune response against Vibrio cholerae in the subject, such as a human. Upon administration of a disclosed composition, the immune system of the subject typically responds to the composition by producing antibodies specific for a pathogen-associated protein. Such a response signifies that an immunologically effective dose was delivered to the subject.

The actual dosage of the composition will vary according to factors, such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimized or improved response.

The disclosed composition (e.g., the fusion protein, nucleic acid or vector encoding the fusion protein, the pharmaceutical composition, the immunogenic composition, or the live attenuated bacterial vaccine) can be used in coordinate (or prime-boost) with vaccination protocols or combinatorial formulations. In certain embodiments, novel combinatorial compositions and coordinate immunization protocols employ separate immunogens or formulations, each directed toward eliciting an anti-pathogen immune response, such as an immune response to proteins from Vibrio cholerae. In specific examples, a combinatorial immunogenic composition can include any of the disclosed compositions (e.g., the fusion protein, nucleic acid or vector encoding the fusion protein, the pharmaceutical composition, the immunogenic composition, or the live attenuated bacterial vaccine) disclosed herein, and an additional fusion protein. In specific examples, the additional fusion protein includes enterotoxigenic Escherichia coli (ETEC) antigens CFA/I/II/IV MEFA, toxoid fusion 3xSTa_{N12S}-mnLT_{R192G/L211A}, and/or Shigella antigens (e.g. SEQ ID NO: 26, 28, and 30). Separate immunogenic compositions that elicit an anti-pathogen immune response can be combined in a polyvalent immunogenic composition administered to a subject in a single immunization step, or they can be administered separately (in monovalent immunogenic compositions) in a coordinate (or prime-boost) immunization protocol.

There can be several boosts, and each boost can be the same or a different disclosed composition (such as a fusion protein including the same epitopes as the initial administration or a fusion protein including one or more different epitopes as the initial administration). In some examples, the boost may be the same composition as another boost or the prime. The prime and boost can be administered as a single dose or multiple doses, for example two doses, three doses, four doses, five doses, six doses, or more can be administered to a subject over days, weeks, or months. Multiple boosts can also be given, such one to five (for example, 1, 2, 3, 4, or 5 boosts) or more. The same or different dosages can be used in a series of sequential immunizations. For example, a relatively large dose can be used in a primary immunization and then a boost with relatively smaller doses.

In some embodiments, the boost can be administered about two, about three, about four, or about four to eight weeks following the prime, or several months after the prime. In some embodiments, the boost can be administered about 5, about 6, about 7, about 8, about 10, about 12, about 18, about 24, about 36, about 48, or about 50 months after the prime, or more or less time after the prime. Periodic additional boosts can also be used at appropriate time points to enhance the subject's “immune memory.” The adequacy of the vaccination parameters chosen, for example, formulation, dose, regimen and the like, can be determined by taking aliquots of serum from the subject and assaying antibody titers during the course of the immunization program. In addition, the clinical condition of the subject can be monitored for the desired effect, for example, prevention of Vibrio cholerae infection or improvement in disease state (for example, reduction in pathogen load or production of protective antibodies). If such monitoring indicates that vaccination is sub-optimal, the subject can be boosted with one or more additional doses, and the administration parameters can be modified in a fashion expected to potentiate the immune response.

The amount of a disclosed composition (e.g., the fusion protein, nucleic acid or vector encoding the fusion protein, the pharmaceutical composition, the immunogenic composition, or the live attenuated bacterial vaccine) that is administered can vary. In some embodiments, the amount administered ranges from about 1-100, 5-50, 1-10, 1-20, 5-15, 5-25, 5-30, 10-50, 10-60, or 25-75 μg/dose (such as administrations via oral, IM, ID, or SC routes). The amount is selected based on the subject population (for example, infant, child, adult, or elderly adult). The amount can be ascertained by standard studies involving observation of antibody titers and other responses in subjects. It is understood that a therapeutically effective amount of a disclosed composition can include an amount that is ineffective at eliciting an immune response by administration of a single dose, but that is effective upon administration of multiple dosages, for example, in a prime-boost administration protocol.

For each particular subject, specific dosage regimens can be evaluated and adjusted over time according to the individual need and professional judgment of the person administering or supervising the administration of the composition (the fusion protein, the nucleic acid encoding the fusion protein, the vector comprising the nucleic acid, the pharmaceutical composition, the immunogenic composition, or the live attenuated bacterial vaccine). The dosage and number of doses will depend on the setting, for example, in an adult or anyone primed by prior Vibrio cholerae infection or immunization, a single dose may be a sufficient booster. In naïve subjects, in some examples, at least two doses are administered for example, at least two or three doses.

In some embodiments, the antibody response of a subject is determined in the context of evaluating effective dosages/immunization protocols. In most instances, it is sufficient to assess the antibody titer in serum or plasma obtained from the subject. Decisions as to whether to administer booster inoculations and/or to change the amount of the therapeutic agent administered to the individual can be at least partially based on the antibody titer level. The antibody titer level can be based on, for example, an immunobinding assay which measures the concentration of antibodies in the serum which bind to a Vibrio cholerae protein or virulence factor.

Determination of effective dosages is typically based on animal model studies followed up by human clinical trials and is guided by administration protocols that significantly reduce the occurrence or severity of targeted disease symptoms or conditions in the subject, or that induce a desired response in the subject (such as a neutralizing immune response). Suitable models in this regard include, for example, murine, rat, rabbit, porcine, feline, ferret, non-human primate, and other accepted animal models. Alternatively, effective dosages can be determined using in vitro models (for example, immunologic and histopathologic assays). Using such models, only ordinary calculations and adjustments are required to determine an appropriate concentration and dose to administer a therapeutically effective amount of the composition (for example, amounts that are effective to elicit a desired immune response or alleviate one or more symptoms of a targeted disease). In alternative embodiments, an effective amount or effective dose of the composition may simply inhibit or enhance one or more selected biological activities correlated with a disease or condition, as set forth herein, for either therapeutic or diagnostic purposes.

Administration of a disclosed composition (e.g., the fusion protein, nucleic acid or vector encoding the fusion protein, the pharmaceutical composition, the immunogenic composition, or the live attenuated bacterial vaccine) to elicit an immune response or to reduce or prevent a Vibrio cholerae infection, can, but does not necessarily, completely prevent or eliminate such an infection, however, is effective so long as the risk of infection is measurably diminished. For example, administration of an effective amount of the composition can decrease Vibrio cholerae infection or risk of infection (for example, as measured by infection of cells or by number or percentage of subjects infected by Vibrio cholerae) by a desired amount, for example, by at least 10%, at least 20%, at least 30%, at least 40%, at least 50%, at least 60%, at least 70%, at least 80%, at least 90%, at least 95%, at least 98%, or even at least 99%, as compared to a suitable control.

In some embodiments, administration of a therapeutically effective amount of one or more of the disclosed compositions to a subject induces a neutralizing immune response in the subject. To assess neutralization activity, following immunization of a subject, serum can be collected from the subject at appropriate time points, frozen, and stored for neutralization testing. Methods to assay for neutralization activity include, but are not limited to microneutralization assays, flow cytometry-based assays, and single-cycle infection assays. In some embodiments, administration of a therapeutically effective amount of one or more of the disclosed compositions to a subject induces production of antibodies in the subject. For example, the production of one or more antibodies reactive one or more target virulence factors, such as Vibrio cholerae CT, TcpA, FlaB, FlaC, FlaD, sialidase (Neu/NanH), LPS, or HlyA. In some embodiments, administration of a therapeutically effective amount reduces colonization (e.g. colonization of the intestinal tract of the host) of Vibrio cholerae in the host, for example, by reducing colonization at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or more.

In certain embodiments, a disclosed composition (e.g., the fusion protein, the nucleic acid or vector encoding the fusion protein, the pharmaceutical composition, the immunogenic composition, or the live attenuated bacterial vaccine) can be administered sequentially with other therapeutic agents, such as anti-Vibrio cholerae, Escherichia coli, and/or Shigella agents (such as an antibiotic; e.g., fluoroquinolones, azithromycin, rifaximin, doxycycline, ciprofloxacin), such as before or after the other agent. Sequential administration includes immediately following, or after an appropriate period of time, such as hours, days, weeks, months, or even years later.

### EXAMPLES

The following examples are provided to illustrate certain particular features and/or embodiments. These examples should not be construed to limit the disclosure to the particular features or embodiments described.

### Example 1

### Materials and Methods

Bacteria and plasmids. Vibrio cholerae and recombinant E. coli strains used in this study are listed in Table 1.

TABLE 1

| Strains | Relevant properties | Sources |
|---|---|---|
| Vibrio cholerae N16961 | O1, El Tor, Inaba | ATCC 39315 |
| Vibrio cholerae O395 | O1, Classical, Ogawa | ATCC 39541 |
| Vibrio cholerae MO45 | O139 (Bengal) | ATCC 51394 |
| Vibrio cholerae El Tor | O3 (non-O1/non-O139) | ATCC 14731 |
| 34-D 23 | | |
| 9746 | ‘cholera MEFA’ in pUC57, DH5α | this study |
| 9747 | ‘cholera MEFA’ in pET28α, DH5α | this study |
| 9748 | ‘cholera MEFA’ in pET28α, BL21(DE3) | this study |
| 9764 | ‘HlyA’ in pET28α, BL21(DE3) | this study |
| 9771 | ‘TcpA (El Tor)’ in pET28α, BL21(DE3) | this study |
| 9779 | ‘FlaC’ in pET28α, BL21(DE3) | this study |
| 9781 | ‘FlaD’ in pET28α, BL21(DE3) | this study |
| 9808 | ‘TcpA (Classical)’ in pET28α, BL21(DE3) | this study |
| 9863 | ‘FlaB’ in pET28α, BL21(DE3) | this study |
| 9866 | ‘NanH (sialidase)’ in pET28α, BL21(DE3) | this study |

Vibrio cholerae serogroup strains acquired from ATCC were used as DNA templates for virulence gene cloning, in vitro antibody protection assays, and rabbit challenge studies. Recombinant Escherichia coli strains expressing virulence genes were to express recombinant proteins as coating antigens in antibody titration ELISAs. V. cholerae strains N16961, 0395, M045 and El Tor 34-D 23 provided by BEI Resources (Manassas, VA) were used as DNA templates to PCR amplify and clone target virulence genes used in in vitro antibody protection assays against bacterial adherence, motility and hemolytic activities, and rabbit challenge studies. Vectors pUC57 (GenScript®, Piscataway, NJ) and pET28u (Novagen®, Madison, WI) were used to clone the cholera MEFA gene and V. cholerae virulence genes tcpA, flaB, flaC, flaD, nanH (neu) and hlyA which were PCR amplified with specific primers. Primers are listed in Table 2. E. coli strain BL21(DE3) (Agilent Technologies®, Santa Clara, CA) was used to express recombinant proteins of the cloned cholera MEFA and V. cholerae virulence genes.

TABLE 2

| | SEQ | | PCR |
|---|---|---|---|
| Pri- | ID | | amplified |
| mers | NO | Sequence (5′-3′) | region |
| TcpA- | 33 | CCCATGGGCATGACATTACTCGAAGTAA | O1 E1 Tor |
| 1 | | TCATTG | and O139 |
| | 34 | TCACGGCCGTTAACTGTTACCAAAAGCT | tcpA |
| | | ACTGTG | subunit |
| | | | gene |
| TcpA- | 35 | CCGCCATGGATGCAATTATTAAAACAGC | O1 Clas- |
| 2 | 36 | TCACGGCCGTTAACTGTTACCAAAAGCT | sical |
| | | ACTG | tcpA |
| | | | subunit |
| | | | gene |
| FlaB | 37 | CTAGCTAGCATGGCAATTAATGTAAACA | flagellin |
| | | CG | B subunit |
| | 38 | TCACGGCCGTTATTATCCCAATAAGCTC | gene |
| | | AGAGCT | |
| FlaC | 39 | CTAGCTAGCATGGCGGTGAATGTAAACA | flagellin |
| | | CC | C subunit |
| | 40 | TCACGGCCGCTAGCCTAGAAGAGCCAGT | gene |
| | | GC | |
| FlaD | 41 | CTAGCTAGCATGGCAGTGAATGTAAATA | flagellin |
| | | C | D subunit |
| | 42 | TCACGGCCGTTATTAACCCAACAGGCTG | gene |
| | | AGTG | |
| NanH | 43 | CGGAATTCCGATGGCAACGGGTGACACT | sialidase |
| | | GAGTT | enzyme |
| | 44 | TCACGGCCGTTATTAGGTATCCCAAGTT | |
| | | ATACC | |
| HlyA | 45 | CCGCCATGGATGCCAAAACTCAATCG | hemolysin |
| | 46 | TCACGGCCGGTTATCGACAGGAAAGGT | gene |

Cholera MEFA gene construction and expression. Selection of a backbone protein and immunodominant B-cell epitopes from target virulence factors, CT, TcpA (of O1 El Tor and Classical), FlaB, FlaC, FlaD, sialidase (Neu/NanH) and HlyA were carried out in silico, by using IEDB epitope prediction program (iedb.org) (Vita et al. (2019) Nucleic Acids Research, 47:D339-D343), protein homology/analogY Recognition Engine (Kelley et al. (2015) Nature Protocols, 10:845-858.), and PyMol (Janson et al. (2017) Bioinformatics, 33:444-446). Additionally, two peptides mimicking O1 LPS specific antigens were also included. Epitope were analyzed for homology among the four V. cholerae strains included in this study as well as strains deposited at NCBI GenBank® database; an additional TcpA epitope was added because of heterogeneity. The virulence factors targeted in the study were those that have been demonstrated to induce protective antibodies against cholera and/or to mount dominant host immune responses after V. cholerae exposure. While the most immunodominant epitope from FlaB backbone was retained, its surface-exposed and less immunodominant epitopes were substituted by the epitopes from CT, TcpA, FlaC/FlaD (an epitope conserved between FlaC and FlaD was identified), sialidase (neuraminidase) and hemolysin A to generate cholera MEFA gene initially in silico. The constructed cholera MEFA was examined for protein structure and stability with PyMol (pymol.org) and ExPASY (expasy.org), and was optimized to position each epitope and to assess epitope native antigenicity by using CHARMM (charmm.org) as previously described (Duan et al. (2017) J Vaccines Vaccin., 8(4):367).

The optimized cholera MEFA gene was synthesized (GenScript) and cloned into vector pUC57, then subcloned into vector pET28u and expressed in E. coli BL21(DE3). Positive colonies were verified with DNA sequencing and then used in protein expression as previously described (Duan et al. (2018) Front Microbiol., 5; 9:1198; Nandre et al. (2016) Vaccine, 34:3620-25; Huang et al. (2018) Applied and Environmental Microbiology, 84:e00849-18). Briefly, a single well-grown colony from the overnight LB/Kan selective agar plate was cultured in 5 mL LB broth containing kanamycin (30 μg/mL) and grown at 37° C. with vigorous shaking (220 rpm) overnight. Overnight growth (3 mL) was transferred to a 500 mL flask with 200 mL 2×Yeast Extract Tryptone (YT; Fisher Scientific®, Waltham, MA) containing kanamycin (30 μg/ml) and grew at 37° C. on a shaker incubator (220 rpm) for approximately 3 hrs, until the OD₆₀₀ reached to 0.5-0.7. Bacteria were then induced with isopropyl β-D-1-thiogalactopyranoside (IPTG; Sigma®, St. Louis, MO; 1 mM) for 4 more hours, and were harvested by centrifugation (13,000×g) at 4° C. for 15 min. Bacteria pellets were frozen overnight at −80, thawed, and incubated with 10 mL bacterial protein extraction reagent (B-PER; Thermo Fisher Scientific®, Rochester, NY). Bacterial suspension after being homogenized with pipetting with a 18G needle and vortexed with a vortex mixer, was incubated at 4° C. on a shaker (120 rpm) for 30 min and sonicated on ice for 5 min. Bacterial lysate was centrifuged (16,200×g) at 4° C. for 15 minutes, and inclusion body proteins were extracted using B-PER by following the manufacturer's protocol.

Inclusion body proteins were solubilized and refolded using one-step refolding reagent and protocol (Novagen®). Briefly, inclusion body protein suspension was incubated with freshly made 1×IB solubilization buffer (50 mM CAPS, pH11.0) supplemented with 0.3% N-lauroylsarcosine and 1 mM DTT at room temperature for 1 hour. Solubilized protein in supernatant was collected with centrifugation (16,200×g) for 10 minutes, transferred to molecular porous membrane tubing (Spectrum Laboratories, Inc., Rancho Dominguez, CA), and dialyzed in 50×dialysis buffer (1M Tris-HCl pH8.5) supplemented with 50 ul 1M DTT (0.1 mM DTT final concentration) at 4° C. for 4 h. After two exchanges, approximately 8 h apart, of dialysis buffer without DTT, refolded protein in clear supernatant was collected and stored at −80° C.

Cholera MEFA protein was characterized in 15% sodium dodecyl sulfate-polyacrylamide gel electrophoresis (SDS-PAGE) with Coomassie blue straining and Western blot using anti-CT and anti-Tcp antisera.

Mouse immunization with cholera MEFA protein. Twenty-four 8-week old female BALB/c mice (Charles River Laboratories International, Inc., Wilmington, MA) in three groups (8 mice per group) were used for cholera MEFA protein intramuscularly (IM) immunization. One group was IM immunized with 25 μg of cholera MEFA protein (in 25 μl PBS), and the second group was IM injected with 25 μg of cholera MEFA protein (in 25 μl PBS) and 0.1 μg adjuvant dmLT (in 1 μl; LTR192G/L211A supplied by PATH). The third group injected with 25 μl of phosphate buffered saline (PBS) served as the control. Two boosters at the same dose as the primary were followed at an interval of two weeks. All mice were euthanized two weeks after the second booster. Blood was collected from the heart of each mouse, and serum samples were stored at −20° C. until use.

Mouse serum antigen-specific antibody titration. Mouse serum IgG antibodies specific to cholera MEFA antigens were titrated in ELISAs. 96-well microtiter 2HB plates (Thermo Fisher Scientific®) coated with CT (Sigma®), TcpA (of O1 El Tor), TcpA (of O1 classical), FlaB, FlaC, FlaD, sialidase (Nan H), HlyA, and O1 LPS O-antigen, 100 ng per well (in 100 μl coating buffer; 15 mM Na₂CO₃, 35 mM NaHCO₃, pH9.6), were incubated 1 h at 37° C., washed with PBST, blocked with 10% skim milk (in PBST), then incubated with two-fold serially diluted serum samples (from 1:200 to 1:256,000) from each mouse 1 h at 37° C., in triplicates. Plate wells were washed, then incubated with horseradish peroxidase (HRP)-conjugated goat-anti-mouse IgG (1:5000; Sigma®) 1 h at 37° C. Wells were washed, incubated with 3,3′,5,5′-tetramethylbenzidine (TMB) microwell peroxidase substrate system (2-C) (KPL, Gaithersburg, MD), and OD₆₅₀ were measured. OD₆₅₀ readings were converted to titers by multiplying the highest serum dilution given an adjusted OD₆₅₀ of more than 0.3 (after subtraction with background OD) with the adjusted OD and expressed in a log₁₀ scale, as described previously (Nandre et al. (2018) Infect Immun., 86:e00550-17; Seo et al. (2020) Hum Vaccin Immunother., 16:419-425; Duan et al. (2018) Front Microbiol., 5; 9:1198; Lu et al. (2019) Appl Environ Microbiol., 85:e00329-19).

Mouse serum antibody neutralization assays. Mouse serum samples were examined for in vitro protection against CT enterotoxicity, CT binding to GM₁ receptor, adherence against O1, O139 and non-O1/non-O139 V. cholerae, V. cholerae motility, and V. cholerae hemolytic activity.

Antibody neutralization against CT enterotoxicity: Cyclic AMP ELISA kit (Enzo Life Sciences, Farmingdale, NY) and T-84 cells (ATCC, #CCL-248™) was used to examine mouse serum antibody neutralization activity against CT enterotoxicity as described previously (Zhang et al. (2010) Infect Immun., 78:316-25; Ruan et al. (2014) Infect Immun., 82:1823-1832). Briefly, T84 cells (1-2×10⁵ per well) cultured in a 24-well BD Falcon® cell culture plate (Fisher Scientific), at 95% to complete confluence, were incubated with 10 ng CT which was premixed with 30 μl mouse serum sample, at a final volume of 1 mL DMEM/F12 medium containing 0.3 mM IBMX, in 5% CO₂ incubator at 37° C. for 3 hrs. Cells were gently but thoroughly washed with PBS to remove extracellular cAMP, lysed with 300 μl of 0.1M HCl with 0.5% Triton X-100 on a shaker (130 rpm) at room temperature for 30 min, collected and centrifugated at 1000×g for 10 min. Intracellular cAMP levels were measured by following manufacturer's protocol (Enzo Life Sciences).

Antibody blocking CT binding to GM₁ receptor: Competitive GM₁ ELISA was used to measure cholera MEFA-induced antibodies for blocking the binding between the CT B pentamer to GM₁. As described previously (Zhang et al. (2010) Infect Immun., 78:316-25; Huang et al. (2018) Applied and Environmental Microbiology, 84:e00849-18), polystyrene 96-well microtiter plates (MaxiSorp™; Nunc, Roskilde, Denmark) coated with GM₁ ganglioside (Sigma; 80 ng per well) overnight at 4° C. were washed with PBST and incubated with 5% skim milk at 37° C. for 1 h. Wells were washed, then incubated with CT (20 ng) premixed with dilutions of mouse serum samples (1:200-1:12,800). After washes with PBST, wells were incubated with anti-CT rabbit antiserum (Sigma; 1:3000), and then goat anti-mouse HRP conjugated antibody (Sigma; 1:5000 dilution) after washes with PBST. OD₆₅₀ was measured with 3,3′,5,5′-tetramethylbenzidine (TMB) microwell peroxidase substrate system (2-C) (KPL) using a plate reader.

Antibody adherence inhibition assay: Mouse serum antibody in vitro protection against V. cholerae adherence was examined Caco-2 cells (ATCC #HTB-37™) as previously described (Seo et al. (2020) Hum Vaccin Immunother., 16:419-425; Ruan et al. (2014) Clin Vaccine Immunol., 21:243-9; Duan et al. (2018) Front Microbiol., 5; 9:1198; Lu et al. (2019) Appl Environ Microbiol., 85:e00329-19). Briefly, V. cholerae strains O1 El Tor N16961, O1 Classical O395, O139 Bengal, or non-O1/non-O139 El Tor 34-D 23 (1.25×10⁴ bacteria) incubated with 15 μl mouse serum sample at room temperature for 30 min on a shaker incubator (50 rpm), were added to 95%-100% confluent Caco-2 cells seeded in a 48-well plate and incubated in 37° C. CO₂ incubator for 1 h. Cells were washed (to remove nonadherent bacteria), dislodged and collected. Collected cells (with adherent bacteria) were suspended, serially diluted and plated at LB agar plates. V. cholerae bacteria (CFUs) grown overnight at 30° C. were counted.

Antibody inhibition of bacterial motility: V. cholerae bacteria at OD 0.1 (10 μl) incubated with mouse or rabbit serum sample (40 μl) for 30 min at room temperature was transferred to a glass slide and examined, using a microscope (Zeiss® Axiovert™ 200M with the Apotome Structured Illumination Optical Sectioning System) at 100× objective and also Axiocam 506™ with high resolution black and white camera to record bacterial motility activity.

Rabbit IM immunization: A total of 20 adult New Zealand White rabbits (Charles River Laboratories International, Inc., Wilmington, MA), four rabbits in a group, were included in rabbit immunization and two challenge studies. In the first study, one group was IM immunized with 200 μg cholera MEFA protein (in 200 μl) and 1 μg dmLT adjuvant (1 μl), the other group was immunized with 200 μl PBS as the control. In the second study, one group was IM immunized with cholera MEFA alone, the second group with cholera MEFA and dmLT, and the third group with PBS as the control. Two booster injections were followed at the same dose of the primary at an interval of two weeks. Serum samples were collected from each rabbit before the primary and every two weeks afterward and stored at −80° C. until use.

Rabbit orogastric challenge with V. cholerae: Two weeks after the second booster injection, each rabbit was orogastrically inoculated with 5×10¹⁰ CFUs (in 1 mL PBS) V. cholerae bacteria. Non-O1/non-O139 strain (34-D 23) was used as the challenge strain for the first study, and O1 El Tor (N16961) was used in the second study. Rabbits were first administered with antacid (famotidine; 0.5-1 mg per kg body weight; IV) via ear vein 3 h prior to inoculation, sedated with dexmedetomidine (0.05-0.25 mg per kg body weight; IM), anesthetized with 3-5% isoflurane, administered with 3 ml 5% sodium bicarbonate with 16′ intermittent red rubber catheter, and then 1 mL bacteria culture (in PBS) and followed with 3 mL sodium bicarbonate. Rabbits were monitored for signs including watery diarrhea and loose feces during 24 h post-inoculation.

Rabbits were euthanized 24 h post-inoculation. Rabbits were first deeply sedated with dexmedetomidine (0.25 mg per kg body; IM), followed with isoflurane, then exsanguinated with cardiac puncture, and finally intracardiac injection of KCl (2 mg/ml). At necropsy, intestines and cecum were examined for fluid accumulation and distal ileal segment (10 cm) was harvested. Cecum content from each rabbit was also collected.

Rabbit antibody titration and antibody in vitro protection: Serum and cecum content suspension samples from each rabbit were examined for IgG and IgA titers to each target virulence factor in ELISAs, as described in above for mouse antibody titration, except the use of HRP-conjugated goat-anti-rabbit IgG and IgA secondary antibodies. Rabbit cecum content suspended in fecal constitution buffer (10 mM tris, 100 mM NaCl, 0.05% Tween®-20, 5 mM sodium azide, pH 7.4; 1 gm cecum in 5 ml buffer) supplemented with 0.5 mM phenylmethylsulfonyl fluoride was centrifuged, and supernatant was collected and used in antibody titration ELISAs.

Rabbit serum samples were examined for in vitro protection against CT enterotoxicity, CT binding to GM₁ receptor, adherence by V. cholerae strains, bacterial motility, vibriocidal, and hemolytic activities as described in above mouse serum antibody neutralization assays. Rabbit cecum content suspension samples were included in bacterial adherence inhibition assay.

Rabbit quantitative small intestine colonization assay, a rabbit colonization model: Distal ileal segment collected from each immunized or control rabbit at necropsy was cut open, thoroughly rinsed to remove fecal content, weighed, and ground in PBS (1 g tissue in 9 mL sterile PBS) in a glass grinder as previously described (Zhang et al. (2010) Infect Immun. 78:316-25). Solution was well mixed, serially diluted, and plated on LB plates. Bacteria (CFUs) grown overnight at 30° C. were counted and recorded. Twenty colonies randomly selected from a plate were PCR amplified with primers specific to TcpA or CT to verify V. cholerae.

Immunization of pregnant rabbits with cholera MEFA protein and challenge of born infant rabbits with V. cholerae: An infant rabbit passive protection model was developed to evaluate cholera MEFA-induced antibodies for cross protection against clinical diarrhea by immunizing pregnant rabbits with the MEFA protein and challenging born infant rabbits with different V. cholerae serotype strains.

Pregnant rabbit immunization: NZW rabbits of timed pregnancy (Charlies River Laboratories) were randomly divided into two groups. Four days after breeding, one group was IM immunized with 250 μg cholera MEFA protein (in 250 μl) and 1 μg dmLT adjuvant (1 μl), the other group with 250 μl PBS and 1 μg dmLT as the control. One booster was followed two weeks later, at the same dose of the primary. Blood was collected before the primary and two weeks after the booster, and milk was collected at necropsy. Blood and milk were examined in ELISAs for IgG and IgA responses to the target virulence factors.

Infant rabbit orogastric challenge with different V. cholerae serogroup strains: After three-day suckling, infant rabbits born from the immunized or the control mother rabbits were anesthetized with IV administration of 50-100 μl famotidine 2-3 h before challenge inoculation. Anesthetized infant rabbits received 250 μl 5% sodium carbonate before orogastric inoculation with V. cholerae O1 El Tor N16961, O1 Classical O395, O139 Bengal, or non-O1/non-O139 (2.5×10⁹ CFUs, in 500 μl), and then 250 μl 5% sodium carbonate after vibrio challenge.

Challenged infant rabbits were monitored every 6 h during the first 12 h and then hourly afterward during 24 h post-inoculation. Clinical outcomes were recorded as cholera or severe diarrhea (profuse diarrhea and dehydration, loss of 20% body weight), watery diarrhea (watery feces), moderate diarrhea (loose and unformed feces), mild diarrhea (semi-formed feces), or normal (with formed fecal pellets). Infant rabbits were euthanized 24 h post-inoculation or earlier if they developed cholera or severe dehydration. All rabbits were measured for body weight before challenge and at 6, 18, 24 h post-inoculation or the time of euthanasia.

At necropsy, fecal formation and fluid accumulation at small intestine, colon and cecum were examined and recorded; blood and ileal distal segment were collected from each infant rabbit. Serum samples were titrated for IgG or IgA responses to the virulence factors targeted by cholera MEFA. Ileal distal segments were homogenized, diluted and plated on plates. After overnight growth at 30° C., CFUs were counted. Twenty colonies were PCR screened with primers specific to TcpA.

Antibody passive protection against clinical cholera or diarrhea assessment: Antibodies in the immunized mothers and the born infant rabbits were titrated. Antibodies levels in the mothers and infants were examined for correlates to clinical protection in infant rabbits, including reduction of V. cholerae intestinal colonization, prevention of weight loss and more importantly clinical diarrhea.

Statistical analyses: Antibody titration, antibody neutralization activity and rabbit colonization data were presented as means and standard deviations. Statistical differences between groups were analyzed using one-way ANOVA. A post hoc (Tukey's test) was used and a calculated p-value of less than 0.05 indicated a significant difference between groups. Significant differences in clinical signs between control and immunized pig groups or protective efficacy was determined with Fisher's exact test.

### Example 2

### Development of the Multivalent Cholera MEFA Protein Antigen

A novel epitope- and structure-based MEFA (multiepitope fusion antigen) vaccinology platform was used to construct a multivalent cholera MEFA protein antigen for broad immunogenicity and cross protective antibodies against cholera. This cholera MEFA selected V. cholerae FlaB, a strong immunogen and an adjuvant, as the backbone to present conserved immunodominant epitopes of CT, TcpA, sialidase, HlyA, flagellins (B, C, D), as well as epitopes mimicking O1 LPS O-antigen domains and epitope native antigenicity. This cholera MEFA protein was then examined for broad immunogenicity in mouse and rabbit immunization. MEFA-derived antibodies for cross protection against adherence from different V. cholerae serogroup strains, neutralization against CT enterotoxicity, blocking CT binding to host receptor GM₁ gangliosides, inhibition against Vibrio motility, vibriocidal and hemolytic activity, were also assessed. Furthermore, rabbits were immunized with this cholera MEFA protein and challenged with a V. cholerae O1 or a non-O1/non-O139 strain. Bacteria colonizing rabbit small intestines was quantified to assess MEFA-induced antibody in vivo protection against V. cholerae colonization and to evaluate the potential application of this multivalent protein antigen for development of an effective injectable cholera vaccine.

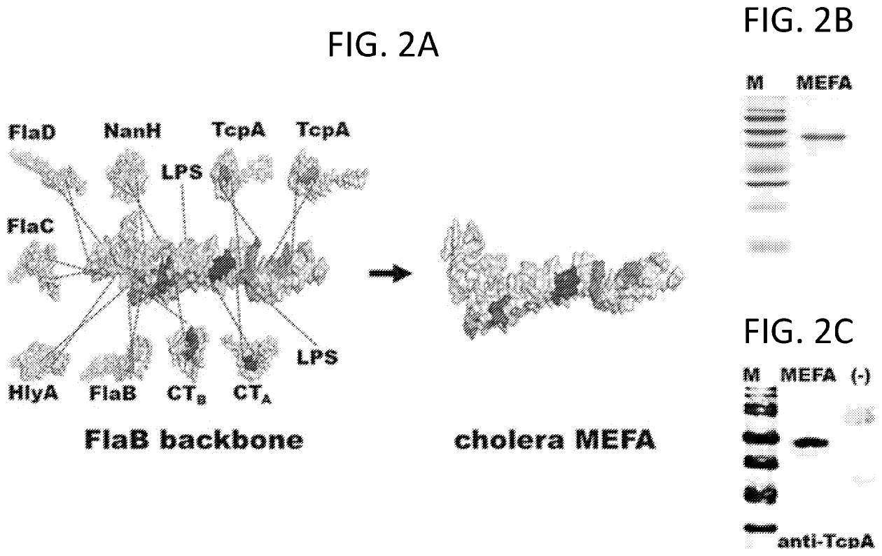

Immunodominant B-cell epitopes from CTA (ADSRPPDEIKQS; SEQ ID NO: 10), CTB (SQHIDSQKKA; SEQ ID NO: 14), TcpA (PATADATAASK of O1 Classical 395 (SEQ ID NO: 8), GKVSADEAKNP of O1 El Tor N16961 and O139 Bengal (SEQ ID NO: 6); the non-O1/non-O139 strain included in this study does not carry TcpA gene), sialidase (MQDNTNNGSGV; SEQ ID NO: 18), FlaB (SNGSNSSSERR; SEQ ID NO: 12), FlaC/D (LQSQSANGSNSKSE, shared by FlaC and FlaD; SEQ ID NO: 22) and HlyA (TGGVEVSGDGPK; SEQ ID NO: 20) were in silico identified and selected as the representative antigens for a cholera MEFA protein. Except two TcpA epitopes which are specific to O1 El Tor or classical biotypes, the other epitopes are homologous among the O1, O139 and non-O1/non-O139 serogroup strains included in this study. Additionally, two epitopes mimicking O1 LPS O-antigen (LPSAGRGVCYEA (SEQ ID NO: 16), QHLNSILLVTK (SEQ ID NO: 24)) were also included (Ghazi and Gargari (2017) Iranian Journal of Microbiology 9:244-250). Flagellin B, which exhibits strong immunogenicity and mucosal adjuvanticity (Lee et al. (2006) Infection and Immunity, 74:694-702), was chosen as the backbone for the construction of this cholera MEFA immunogen (FIG. 2A). Cholera MEFA protein was in silico optimized to have each epitope surface exposed and to retain epitope antigenicity. This cholera MEFA gene was then synthesized; the synthetic gene was inserted into plasmid pUC57 initially and subcloned into expression vector pET28a. Cholera MEFA protein (39.2 kDa) expressed by E. coli strain BL21 (DE3) was extracted and refolded at a yield of 40 mg per liter of culture broth and a purity of greater than 90% estimated based on Coomassie blue staining (FIG. 2B). MEFA protein was verified in Western blot with mouse anti-TcpA antiserum and rabbit anti-CT antiserum (FIG. 2C).

### Example 3

### Cholera MEFA Protein Induced Broad Immune Responses in Mice

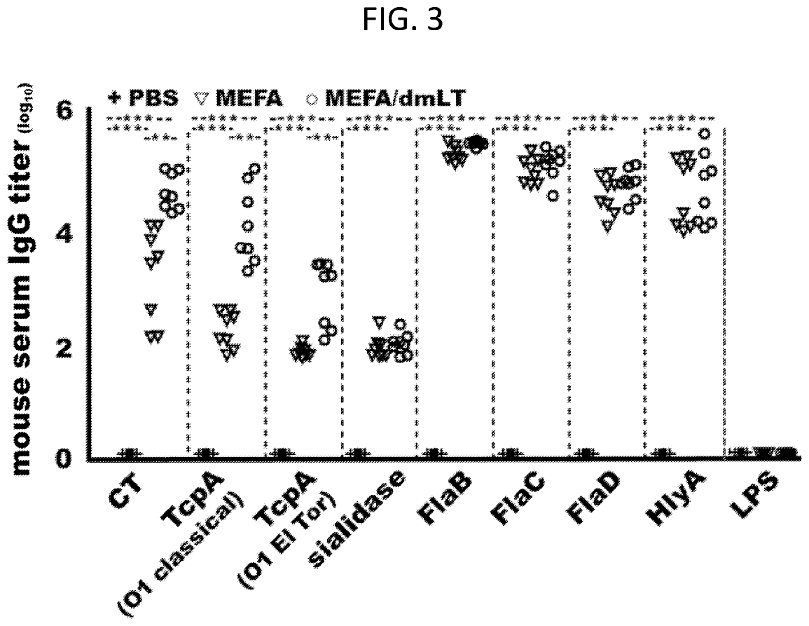

Mice that were intramuscularly (IM) immunized with cholera MEFA protein, with or without adjuvant dmLT, developed antigen-specific IgG responses (FIG. 3). Anti-CT, -TcpA (O1 Classical), -TcpA (O1 El Tor & O139), -sialidase, -FlaB, -FlaC, -FlaD and anti-HlyA IgG titers were 3.3±0.84, 2.3±0.32, 1.9±0.09, 2.0±0.20, 5.4±0.13, 5.1±0.23, 4.7±0.34, and 4.7±0.60 (log₁₀), respectively, from serum samples of the group immunized with the MEFA protein. The IgG titers to CT, TcpA (O1 Classical), TcpA (O1 El Tor & O139), sialidase, FlaB, FlaC, FlaD and to HlyA were 4.8±0.33, 4.2±0.68, 3.0±0.58, 2.1±0.18, 5.6±0.05, 5.3±0.28, 4.9±0.26 and 4.8±0.63 (log₁₀), respectively, in the mice immunized with cholera MEFA protein adjuvanted with dmLT. Anti-LPS O1 antigen IgG response was not detected from the mouse serum samples. No antigen-specific IgG responses were detected from the control mouse serum samples.

Mice IM immunized with cholera MEFA protein adjuvanted with dmLT developed greater anti-CT and anti-TcpA IgG responses than the mice immunized with cholera MEFA protein alone (p<0.01) (FIG. 3); whereas the IgG titers to the other antigens were not significantly different between the two immunized groups.

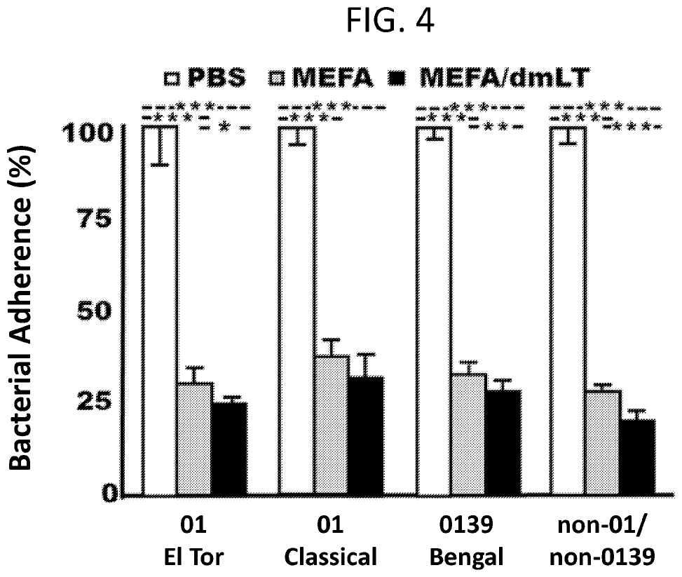

Vibrio cholerae O1 El Tor strain N16961, O1 Classical strain 395, O139 strain M045, and non-O1 and non-O139 strain El Tor 34-D 23, after incubation with the heat-inactivated mouse serum samples pooled from the group IM immunized cholera MEFA or cholera MEFA and dmLT adjuvant, showed a significant reduction at adherence to Caco-2 cells (FIG. 4). Mouse serum samples from the group immunized with cholera MEFA, with (grey) or without (black) adjuvant dmLT, showed the same level of antibody neutralization activity against adherence of O1 El Tor and Classical strains, whereas the group with dmLT adjuvant were significantly better in inhibiting adherence of O139 and non-O1 and non-O139 strains. Antibody adherence inhibition results indicated cholera MEFA-induced anti-TcpA antibodies are neutralizing. Incubated with the serum from the mice immunized with cholera MEFA, V. cholerae O1 El Tor N19691, O1 classical 395, O139 Bengal or non-O1/non-O139 34 D-23 bacteria (CFUs) adherence to Caco-2 cells were reduced by 70%, 62%, 67%, and 72% respectively, compared to the bacteria incubated with the serum from the group injected with PBS. Treated with the sera from the mice immunized with the MEFA protein and adjuvant dmLT, adherence of the four serogroup strains (CFUs) was decreased by 74%, 68%, 72%, and 80%, respectively.

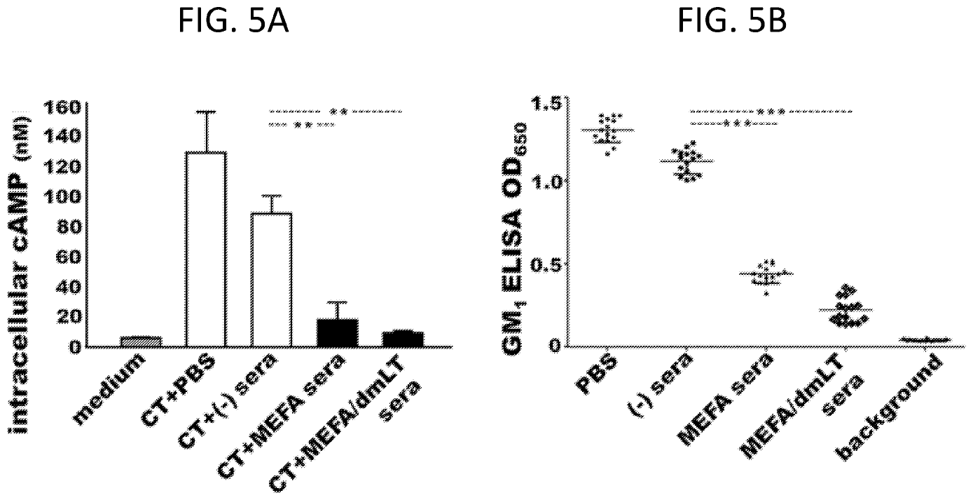

Cholera MEFA-induced mouse serum antibodies neutralized CT enterotoxicity. Mouse serum samples from the group immunized with cholera MEFA, with or without adjuvant dmLT, prevented CT from elevating intracellular cyclic adenoses phosphatase (cAMP) in T-84 cells (FIG. 5A). This demonstrated that cholera MEFA-induced antitoxin antibodies neutralized CT enterotoxicity. The intracellular cAMP levels in T-84 cells incubated with CT premixed the serum from mice immunized with cholera MEFA or cholera MEFA and dmLT adjuvant were 17.5±16.8 and 8.3±2.2 pMol, respectively, which were significantly lower than the cAMP in cells incubated with CT and the control mouse serum (88.5±16.8 pMol; p<0.01).

Cholera MEFA-induced mouse serum antibodies blocked CT binding to GM₁ gangliosides.

GM₁ competitive ELISA showed that CT, after incubation with the serum from the mice immunized with cholera MEFA or cholera MEFA and dmLT adjuvant, exhibited a significant reduction at binding to GM₁ gangliosides (FIG. 5B). The OD₆₅₀ readings in the GM₁-coated wells were 0.44±0.05 and 0.22±0.08, respectively, after incubation with CT premixed with the mouse serum from the group immunized with cholera MEFA protein alone or along with dmLT adjuvant. These OD values were significantly lower than the OD in the wells incubated with CT premixed with the control mouse serum (1.12±0.07; p<0.001) or PBS (1.30±0.07; p<0.001).

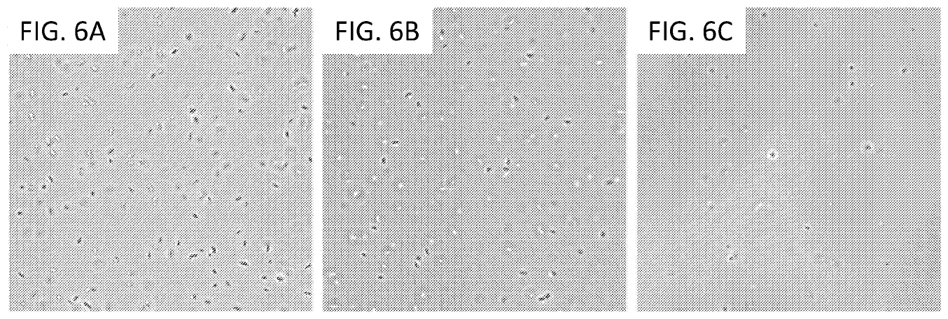

Cholera MEFA-induced mouse serum antibodies halted V. cholerae motility. Microscopic examination observed that V. cholerae O1 El Tor N19691, O1 classical 395, O139 Bengal and non-O1/non-O139 34 D-23 bacterial motility was halted when the mouse serum samples from the immunized group, cholera MEFA protein with or without dmLT adjuvant, were added to the bacteria suspension. Motility of these bacteria were not affected with the addition of the control mouse serum. That indicated that the anti-flagellin antibodies induced by the cholera MEFA protein were functional to prevent motility of various V. cholerae strains (FIGS. 6A-6C).

Vibriocidal antibody titers and antibody activity against hemolysis were not detected in mouse serum samples. The OD₅₉₅ values in the wells with V. cholerae O1 El Tor N19691 incubated with mouse sera of the immunized or the control group were 0.32±0.07 and 0.33±0.03, respectively, and showed no reduction compared to the OD₅₉₅ from the growth control well (0.24±0.04). Colonies of V. cholerae O1 El Tor N19691, O1 classical 395, O139 Bengal or non-O1/non-O139 34 D-23 displayed no morphological alteration on sheep blood agar plates coated with mouse sera from the group immunized with the cholera MEFA or PBS.

### Example 4

Cholera MEFA protein induced antigen-specific and protective antibodies in rabbit Rabbits IM immunized with cholera MEFA were protected against Vibrio cholerae challenge. New Zealand white rabbits (1.5-2.5 kg) were IM immunized with cholera MEFA (with dmLT adjuvant). After challenge with non-O1 and non-O139 strain, two of the four control rabbits developed mild diarrhea. All of the immunized rabbits remained healthy.

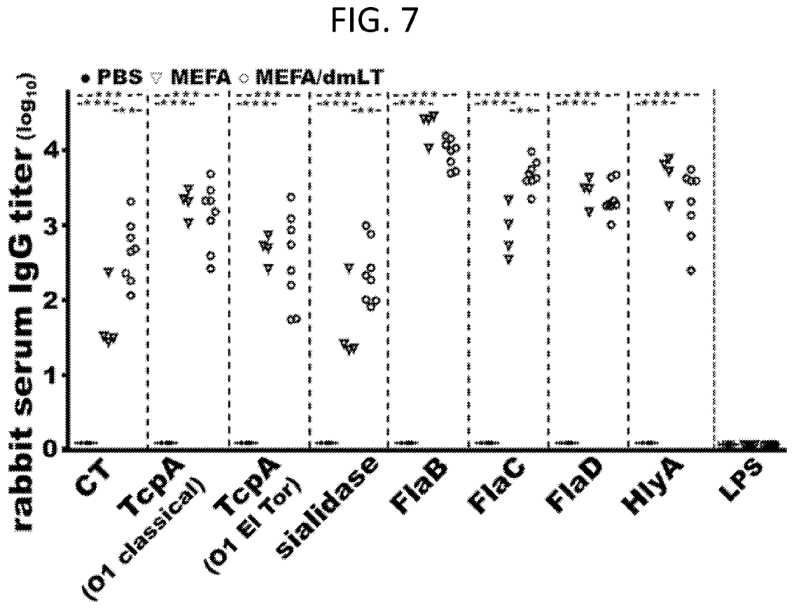

Rabbits IM immunized with cholera MEFA developed broad antibody responses to the target antigens (FIG. 7). Rabbit anti-CT, -TcpA (O1 classical), -TcpA (O1 El Tor and O139), -sialidase, -FlaB, -FlaC, -FlaD and anti-HlyA IgG titers (log₁₀) were 1.7±0.44, 3.3±0.19, 2.7±0.19, 1.6±0.53, 4.3±0.20, 2.9±0.35, 3.5±0.20, and 3.7±0.28, respectively, in the serum samples of the group IM immunized with cholera MEFA protein. IgG titers in the serum of the rabbits IM immunized with cholera MEFA and dmLT adjuvant were 2.6±0.41, 3.1±0.43, 2.5±0.61, 2.4±0.40, 4.0±0.19, 3.7±0.19, 3.3±0.22 and 3.3±0.47 (log₁₀), respectively. No antigen specific antibodies were detected from the control rabbits.

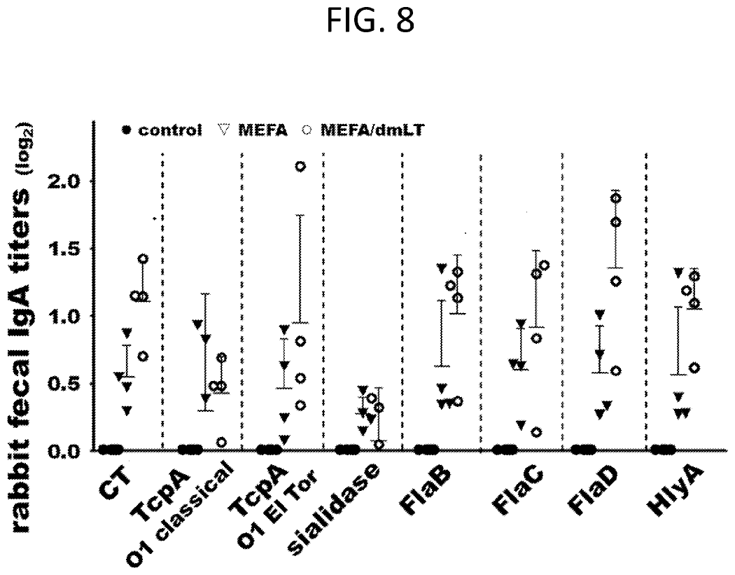

Mild antigen-specific IgA antibodies were detected in the cecum contents of the immunized rabbits (FIG. 8). Anti-CT, -TcpA (O1 classical), -TcpA (O1 El Tor and O139), -FlaB, -FlaC, -FlaD and anti-HlyA IgA titers (in log 2) were 0.54±0.24, 0.30±0.87, 0.46±0.37, 0.62±0.49, 0.60±0.31, 0.58±0.34, and 0.57±0.50, respectively, from the cecum content suspensions of the rabbits IM immunized with cholera MEFA, and were 1.1±0.30, 0.43±0.26, 0.95±0.79, 1.0±0.43, 0.95±0.57, 1.4±0.57, and 1.0±0.30 (log 2), respectively, in the cecum contents of the rabbits IM immunized with cholera MEFA and dmLT adjuvant. Anti-sialidase IgA was not detectable from either immunized group. No antigen-specific IgA was detected from the control rabbit cecum contents.

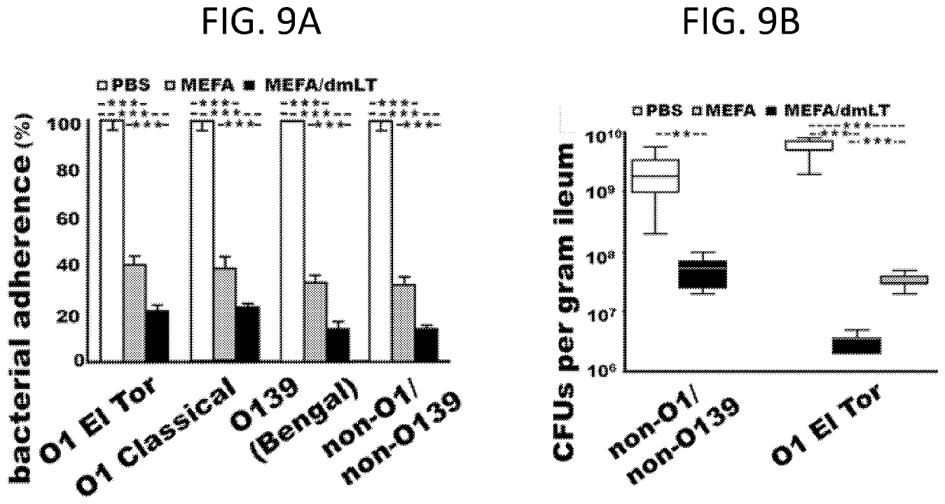

Rabbit serum samples from the group immunized with cholera MEFA protein, with or without dmLT adjuvant, neutralized CT enterotoxicity, blocked CT binding to GM₁, blocked V. cholerae bacterial motility. Additionally, the serum (and cecum content suspension) samples from the rabbit IM immunized with cholera MEFA (with or without dmLT adjuvant) significantly inhibited adherence of V. cholerae O1 El Tor N19691, O1 classical 395, O139 Bengal and non-O1/non-O139 34 D-23 to Caco-2 cells (FIG. 9A). Rabbit serum pooled from the immunized group or from individual rabbits showed no differences in inhibiting V. cholerae adherence. Similarly, after incubation with rabbit serum samples from the group immunized with cholera MEFA and dmLT adjuvant, or the cecum content suspension samples from the immunized rabbits, V. cholerae O1 El Tor N19691, O1 classical 395, O139 Bengal and non-O1/non-O139 34 D-23 adherence to Caco-2 cells was significantly reduced.

Rabbits IM immunized with cholera MEFA protein, with or without dmLT adjuvant, showed a significant reduction in colonization by Vibrio cholerae O1 El Tor N16961 or a non-O1/non-O139 strain in small intestines (FIG. 9B). Rabbits remained normal and showed no diarrhea or fluid accumulation in intestine or caecum after orogastric inoculation with strain N16961. V. cholerae O1 El Tor N16961 bacteria recovered from the ground distal ileal segment (CFUs per gram) from the rabbits immunized with cholera MEFA alone or cholera MEFA and dmLT adjuvant were (3.4±0.88)×10⁷ and (3.0±0.89)×10⁶, respectively, significantly less than the bacteria colonized in the control rabbits (5.5±1.54)×10⁹ CFUs (p<0.001). Colonies were 100% positive of V. cholerae based on PCR screening with TcpA-specific primers.

When non-O1/non-O139 strain El Tor 34-D 23 was used as the challenge strain, the rabbits IM immunized with cholera MEFA and dmLT adjuvant remained healthy and had (4.0±1.6)×10⁷ CFUs V. cholerae isolated per gram of distal ileal segment. In contrast, two of the four control rabbits shed loose feces and had yellow fluid accumulated in small intestine and caecum. The control rabbits had nearly two-log more V. cholerae colonized in ileum [(2.2±1.5)×10⁹ CFUs per gram; p<0.01].

### Example 5

Protection of Infant Rabbits Born to Mothers IM Immunized with Cholera MEFA

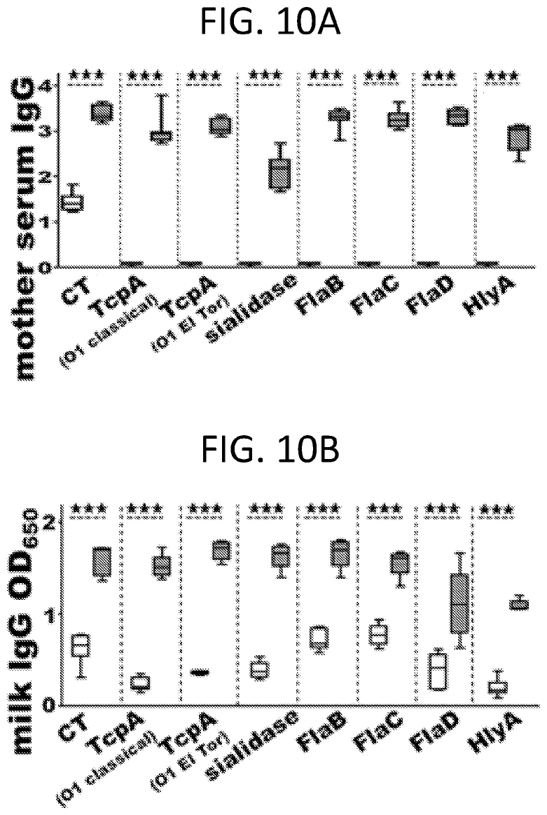

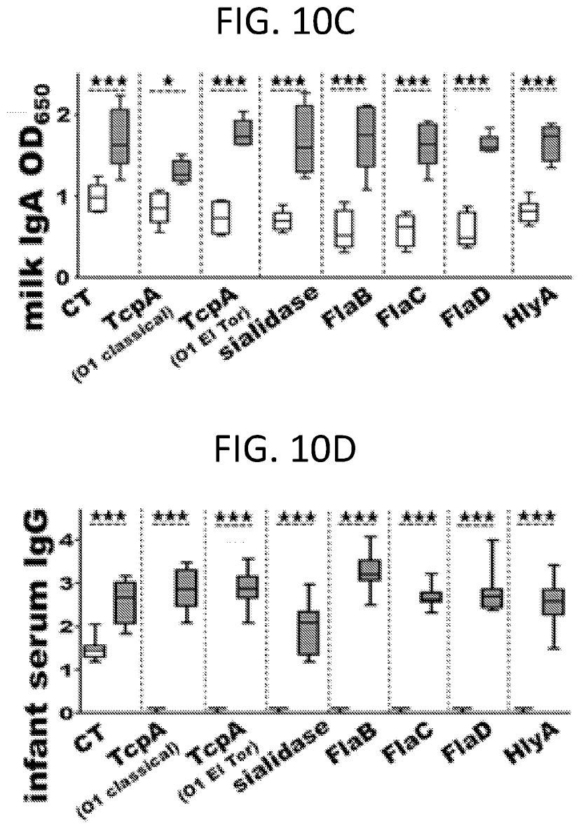

Thirteen rabbits of timed pregnancy were included to reproduce infant rabbits for assessing preclinical efficacy of cholera MEFA-derived antibodies against clinical diarrhea from different V. cholerae serogroup strains. With two immunized rabbits showing false pregnancy, five immunized (with cholera MEFA protein and dmLT adjuvant) and six control (PBS and dmLT adjuvant) mothers delivered 34 and 40 infant rabbits, respectively. Robust IgG (titers in log₁₀) in serum samples and moderate IgG and IgA (OD₆₅₀ values were shown due to high background) in milk samples to all target virulence factors except LPS were detected from the mother rabbits IM immunized with two doses of cholera MEFA protein and adjuvant dmLT (FIGS. 10A-10C). Antigen-specific IgG antibodies (except anti-LPS) were detected from the serum of the infant rabbits born to the immunized mothers (FIG. 10D). In contrast, no antigen-specific IgG or IgA antibodies except moderate anti-CT serum IgG were detected from the control rabbits which were IM injected with PBS and dmLT adjuvant or their born infant rabbits.

Infant rabbits born to the immunized mothers, or the control mothers, were divided into four groups and challenged with V. cholerae O1 El Tor N16961, O1 Classical O395 strain, O139 Bengal strain, or non-O1/non-O139 El Tor 34-D 23 strain. After orogastric inoculation, the infant rabbits born to the immunized mothers were protected from diarrhea. Only 3 (out of 15 challenged with V. cholerae El tor N16961) and 1 (out of 6 inoculated with O139 Bengal strain) infant rabbits born to immunized mothers developed mild diarrhea. In contrast, all infant rabbits (n=40) born to the control mothers (IM immunized with PBS and dmLT adjuvant) developed diarrhea (6 with mild diarrhea and 34 with severe diarrhea) after challenge with the four serogroup strains (Table 3). The efficacy for cross protection against any diarrhea and severe diarrhea from these four V. cholerae strains was 88.4% and 100%, respectively, with 80%, 100%, 83.3%, and 100% efficacy against any diarrhea from V. cholerae O1 El Tor N16961, O1 Classical O395 strain, O139 Bengal strain, or non-O1/non-O139 El Tor 34-D 23 strain individually.

Additionally, during the 24 hours post challenge, infant rabbits born to the immunized mothers showed a positive weight gain, whereas the infant rabbits born to the control mothers lost body weight (Table 3). Overall, infant rabbits with passive antibodies gained 3.1±2.5(%) body weight after V. cholerae challenge, significantly higher than the infant rabbits born to the control mothers, which all lost weight after challenge (−17.6±2.8; p<0.001). Furthermore, colonized vibrio bacteria in the small intestines from infant rabbits born to the immunized mothers were 2 logs fewer than the infant rabbits born to the control mothers, reduced by 99.9%, 99.7%, 99.8% and 99.9% respectively for challenge strain V. cholerae O1 El Tor N16961, O1 Classical O395 strain, O139 Bengal strain or non-O1/non-O139 El Tor 34-D 23 (Table 3).

TABLE 3

| | clinical outcome | | | | | | |
|---|---|---|---|---|---|---|---|
| | no of rabbits with clinical outcome | | | % efficacy against | | infant rabbit | V. cholerae |
| | | mild | severe | any | severe | daily wt. gain | colonized at small |
| infant rabbits | healthy | diarrhea | diarrhea | diarrhea | diarrhea | (%) | intestine (CFUs) |

challenged with O1 El Tor N16961

| | | | | | | | |
|---|---|---|---|---|---|---|---|
| immunized | 12 | 3 | 0 | 80 | 100 | 1.54 ± 3.64*** | 2.4 ± 1.6 (×10⁶)*** |
| (n = 15) | | | | | | | |
| control (n = 16) | 0 | 2 | 14 | | | −18.0 ± 3.92 | 1.7 ± 0.7 (×10⁹) |

challenged with O1 Classical O395

| | | | | | | | |
|---|---|---|---|---|---|---|---|
| immunized | 7 | 0 | 0 | 100 | 100 | 6.84 ± 6.51*** | 1.0 ± 1.3 (×10⁶)*** |
| (n = 7) | | | | | | | |
| control (n = 9) | 0 | 0 | 9 | | | −17.92 ± 6.05 | 4.0 ± 1.7 (×10⁸) |

challenged with O139 (Bengal)

| | | | | | | | |
|---|---|---|---|---|---|---|---|
| immunized | 5 | 1 | 0 | 83.3 | 100 | 1.89 ± 1.49*** | 1.9 ± 0.6 (×10⁶)** |
| (n = 6) | | | | | | | |
| control (n = 8) | 0 | 3 | 5 | | | −20.57 ± 8.43 | 7.8 ± 6.9 (×10⁸) |

challenged with non-O1/non-O139 El Tor 34-D 23

| | | | | | | | |
|---|---|---|---|---|---|---|---|
| immunized | 6 | 0 | 0 | 100 | 100 | 2.16 ± 1.00*** | 1.5 ± 0.9 (×10⁶)* |
| (n = 6) | | | | | | | |
| control (n = 7) | 0 | 1 | 6 | | | −13.91 ± 6.64 | 2.5 ± 4.6 (×10⁹) |

### Example 6

Cholera MEFA is Antigenically Compatible with ETEC Antigens

Mice IM immunized with cholera MEFA and ETEC CFA/I/II/IV developed IgG antibodies to cholera TcpA, CT, sialidase, HlyA, FlaB, FlaC and FlaD and to ETEC CFA/I, CS1, CS2, CS3, CS4, CS5 and CS6. Mice co-immunized with cholera MEFA and ETEC toxoid fusion 3xSTa_{N12S}-mnLT_{R192G/L211A} developed IgG antibodies to TcpA, CT (or LT), sialidase, HlyA, FlaB, FlaC, FlaD and STa. Furthermore, mice IM immunized with three proteins, cholera MEFA, ETEC CFA/I/II/IV and ETEC toxoid fusion 3xSTa_{N12S}-mnLT_{R192G/L211A} developed IgG antibodies to TcpA, CT (or LT), sialidase, HlyA, FlaB, FlaC and FlaD, CFA/I, CS1, CS2, CS3, CS4, CS5, CS6 and STa. These results indicate that this cholera MEFA protein can be combined with ETEC proteins for the development of a combination injectable subunit vaccine against cholera and ETEC diarrhea.

The cholera MEFA can be combined with two ETEC proteins (CFA/I/II/IV MEFA, toxoid fusion 3xSTa_{N12S}-mnLT_{R192G/L211A}) and Shigella MEFA for a combination vaccine against three groups of enteric pathogens—Vibrio cholerae, ETEC and Shigella spp.

These data collectively demonstrate that the multivalent cholera MEFA protein was strongly immunogenic and MEFA-induced antibodies were broadly protective. The antibodies also demonstrated in vitro neutralization against CT enterotoxicity, blocking CT binding to GM1 receptor, abolishment of V. cholerae motility, inhibition of adherence from O1, O139 and non-O1/non-O139 serogroups to Caco-2 cells.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A fusion protein, comprising:
a backbone protein, wherein the backbone protein comprises a consensus sequence having at least 90% sequence identity to SEQ ID NO: 4, and at least one heterologous Vibrio cholerae epitope.

2. The fusion protein of claim 1, wherein the backbone protein comprises or consists of SEQ ID NO: 4.

3. The fusion protein of claim 1, wherein the at least one heterologous Vibrio cholerae epitope comprises 8-15 amino acids.

4-5. (canceled)

6. The fusion protein of claim 1, wherein the Vibrio cholerae epitope comprises a peptide of one or more of: toxin coregulated pilus A (TcpA), cholera toxin (CTA), cholera toxin subunit B (CTB), LPS O-antigen mimic (LPS), sialidase (Neu), hemolysin A (HlyA), flagellin C (FlaC), and flagellin D (FlaD).

7. The fusion protein of claim 6, wherein the at least one heterologous Vibrio cholerae epitope comprises one or more of SEQ ID NOs: 6, 8, 10, 14, 16, 18, 20, 22, 24, 31, and 32.

8. The fusion protein of claim 1, further comprising at least one homologous V. cholerae epitope.

9. The fusion protein of claim 8, wherein the at least one homologous V. cholerae epitope comprises a flagellin B subunit (FlaB) epitope.

10. The fusion protein of claim 9, wherein the at least one homologous V. cholerae epitope comprises SEQ ID NO: 12.

11. The fusion protein of claim 1, wherein the fusion protein comprises each of SEQ ID NOs: 6, 8, 10, 12, 14, 16, 18, 20, 22, and 24; or each of SEQ ID NOs: 6, 8, 10, 12, 14, 18, 20, 22, 31, and 32.

12. The fusion protein of claim 1, comprising at least two V. cholerae epitopes, wherein at least one of the two epitopes is from a different V. cholerae serogroup or biotype.

13. (canceled)

14. The fusion protein of claim 1, wherein the fusion protein comprises an amino acid sequence having at least 90% sequence identity to SEQ ID NO: 2, or an amino acid sequence comprising or consisting of SEQ ID NO: 2.

15. (canceled)

16. A nucleic acid encoding the fusion protein of claim 1.

17-18. (canceled)

19. The nucleic acid of claim 16, wherein the nucleic acid comprises a sequence having at least 90% sequence identity to SEQ ID NO: 1, or the nucleic acid comprises or consists of SEQ ID NO: 1.

20-22. (canceled)

23. A vector, comprising the nucleic acid of claim 16.

24-26. (canceled)

27. An isolated host cell transformed with the vector of claim 23.

28. A pharmaceutical composition comprising the fusion protein of claim 1 or a nucleic acid encoding the fusion protein; and a pharmaceutically acceptable carrier.

29. The pharmaceutical composition of claim 28, further comprising an adjuvant.

30-32. (canceled)

33. A live attenuated bacterial vaccine, comprising the nucleic acid of claim 16, wherein the nucleic acid is expressed in Ty21a.

34. A method of inducing an immune response to Vibrio cholerae in a subject, comprising administering to the subject an effective amount of the pharmaceutical composition of claim 28.

35-38. (canceled)

39. The method of claim 34, wherein the pharmaceutical composition is administered orally, subcutaneously (SC), intramuscularly (IM), or intradermally (ID).

40. The method of claim 34, wherein the subject is human.


SEQUENCE LISTING (46 sequences)

SEQ ID NO: 1 - DNA, 1134 bp, Artificial Sequence
  Exemplary nucleic acid sequence encoding a cholera MEFA fusion protein
atgggcgcgattaacgtgaacaccaacgtgagcgcgatgaccggcaaagtgagcgcggatgaagcgaaaaacccgatgcagaaaagcatggaacgcctgagcccggcgaccgcggatgcgaccgcggcgagcaaagcggcgggcctgcagattagcaaccgcctggcggatagccgcccgccggatgaaattaaacagagcaacgcgaacgatggcattagcattgcgcagaccgcggaaggcgcgatgaacgaaaccaccaacattctgcagcgcatgcgcgatctggcgctgcagagcagcaacggcagcaacagcagcagcgaacgccgcgcgattcaggaaagccagcatattgatagccagaaaaaagcgcgcattgcggaaaccaccagctttggcggcctgccgagcgcgggccgcggcgtgtgctatgaagcgtttcagattggcgcggatagcggcgaagcggtgatgctgagcatgggcagcatgcgcagcgatacccaggcgatgggcggcaaaagctatcgcgcgcaggaaggcaaaatgcaggataacaccaacaacggcagcggcgtgctgaccctgagctataccaacaaacagggcgaagcgcgcaccggcggcgtggaagtgagcggcgatggcccgaaagaagaactggcgacctatattaacggccagaccgaagatgtgaaagcgagcgtgggcgaagatggcaaactgctgcagagccagagcgcgaacggcagcaacagcaaaagcgaaattggcggcggcctgggcggcgaaattggctttgatgcgggccgcaacgtgaccgtggcggatgtgaacgtgagcaccgtggcgggcagccaggaagcggtgagcattctggatggcgcgctgaaagcggtggatagccagcgcgcgagcctgggcgcgtttcagaaccgctttggccatgcgattagcaacctggataacgtgaacgaaaacgtgaacgcgagccgcagccgcattcgcgataccgattatcagcatctgaacagcattctgctggtgaccaaaattctgcagcaggcgagcaccagcgtgctggcgcaggcgaaacagagcccgagcgcggcgctgagcctgctgggctaa

SEQ ID NO: 2 - protein, 377 aa, Artificial Sequence
  Exemplary cholera MEFA fusion protein
MGAINVNTNVSAMTGKVSADEAKNPMQKSMERLSPATADATAASKAAGLQISNRLADSRPPDEIKQSNANDGISIAQTAEGAMNETTNILQRMRDLALQSSNGSNSSSERRAIQESQHIDSQKKARIAETTSFGGLPSAGRGVCYEAFQIGADSGEAVMLSMGSMRSDTQAMGGKSYRAQEGKMQDNTNNGSGVLTLSYTNKQGEARTGGVEVSGDGPKEELATYINGQTEDVKASVGEDGKLLQSQSANGSNSKSEIGGGLGGEIGFDAGRNVTVADVNVSTVAGSQEAVSILDGALKAVDSQRASLGAFQNRFGHAISNLDNVNENVNASRSRIRDTDYQHLNSILLVTKILQQASTSVLAQAKQSPSAALSLLG

SEQ ID NO: 3 - DNA, 1134 bp, Artificial Sequence
  Exemplary nucleic acid sequence of a backbone protein
  misc_feature (43)..(75): “N” is any nucleic acid
  misc_feature (103)..(135): “N” is any nucleic acid
  misc_feature (166)..(201): “N” is any nucleic acid
  misc_feature (301)..(333): “N” is any nucleic acid
  misc_feature (346)..(375): “N” is any nucleic acid
  misc_feature (406)..(441): “N” is any nucleic acid
  misc_feature (550)..(582): “N” is any nucleic acid
  misc_feature (622)..(657): n is a, c, g, or t
  misc_feature (730)..(772): “N” is any nucleic acid
  misc_feature (1024)..(1056): “N” is any nucleic acid
atgggcgcgattaacgtgaacaccaacgtgagcgcgatgaccnnnnnnnnnnnnnnnnnnnnnnnnnnnnnnnnnatgcagaaaagcatggaacgcctgagcnnnnnnnnnnnnnnnnnnnnnnnnnnnnnnnnngcggcgggcctgcagattagcaaccgcctgnnnnnnnnnnnnnnnnnnnnnnnnnnnnnnnnnnnnaacgcgaacgatggcattagcattgcgcagaccgcggaaggcgcgatgaacgaaaccaccaacattctgcagcgcatgcgcgatctggcgctgcagagcnnnnnnnnnnnnnnnnnnnnnnnnnnnnnnnnngcgattcaggaannnnnnnnnnnnnnnnnnnnnnnnnnnnnncgcattgcggaaaccaccagctttggcggcnnnnnnnnnnnnnnnnnnnnnnnnnnnnnnnnnnnntttcagattggcgcggatagcggcgaagcggtgatgctgagcatgggcagcatgcgcagcgatacccaggcgatgggcggcaaaagctatcgcgcgcaggaaggcaaannnnnnnnnnnnnnnnnnnnnnnnnnnnnnnnnctgaccctgagctataccaacaaacagggcgaagcgcgcnnnnnnnnnnnnnnnnnnnnnnnnnnnnnnnnnnnngaagaactggcgacctatattaacggccagaccgaagatgtgaaagcgagcgtgggcgaagatggcaaactgnnnnnnnnnnnnnnnnnnnnnnnnnnnnnnnnnnnnnnnnnnattggcggcggcctgggcggcgaaattggctttgatgcgggccgcaacgtgaccgtggcggatgtgaacgtgagcaccgtggcgggcagccaggaagcggtgagcattctggatggcgcgctgaaagcggtggatagccagcgcgcgagcctgggcgcgtttcagaaccgctttggccatgcgattagcaacctggataacgtgaacgaaaacgtgaacgcgagccgcagccgcattcgcgataccgattatnnnnnnnnnnnnnnnnnnnnnnnnnnnnnnnnnattctgcagcaggcgagcaccagcgtgctggcgcaggcgaaacagagcccgagcgcggcgctgagcctgctgggctaa

SEQ ID NO: 4 - protein, 377 aa, Artificial Sequence
  Exemplary backbone protein
  MISC_FEATURE (15)..(25): “Xaa” is any amino acid
  MISC_FEATURE (35)..(45): “Xaa” is any amino acid
  MISC_FEATURE (56)..(67): “Xaa” is any amino acid
  MISC_FEATURE (101)..(111): “Xaa” is any amino acid
  MISC_FEATURE (116)..(125): “Xaa” is any amino acid
  MISC_FEATURE (136)..(147): “Xaa” is any amino acid
  MISC_FEATURE (184)..(194): “Xaa” is any amino acid
  MISC_FEATURE (208)..(219): “Xaa” is any amino acid
  MISC_FEATURE (244)..(257): “Xaa” is any amino acid
  MISC_FEATURE (342)..(352): “Xaa” is any amino acid
MGAINVNTNVSAMTXXXXXXXXXXXMQKSMERLSXXXXXXXXXXXAAGLQISNRLXXXXXXXXXXXXNANDGISIAQTAEGAMNETTNILQRMRDLALQSXXXXXXXXXXXAIQEXXXXXXXXXXRIAETTSFGGXXXXXXXXXXXXFQIGADSGEAVMLSMGSMRSDTQAMGGKSYRAQEGKXXXXXXXXXXXLTLSYTNKQGEARXXXXXXXXXXXXEELATYINGQTEDVKASVGEDGKLXXXXXXXXXXXXXXIGGGLGGEIGFDAGRNVTVADVNVSTVAGSQEAVSILDGALKAVDSQRASLGAFQNRFGHAISNLDNVNENVNASRSRIRDTDYXXXXXXXXXXXILQQASTSVLAQAKQSPSAALSLLG

SEQ ID NO: 5 - DNA, 33 bp, Artificial Sequence
  Exemplary nucleic acid encoding a TcpA epitope
ggcaaagtgagcgcggatgaagcgaaaaacccg

SEQ ID NO: 6 - protein, 11 aa, Artificial Sequence
  Exemplary TcpA epitope
GKVSADEAKNP

SEQ ID NO: 7 - DNA, 33 bp, Artificial Sequence
  Exemplary nucleic acid sequence encoding a TcpA epitope
ccggcgaccgcggatgcgaccgcggcgagcaaa

SEQ ID NO: 8 - protein, 11 aa, Artificial Sequence
  Exemplary TcpA epitope
PATADATAASK

SEQ ID NO: 9 - DNA, 36 bp, Artificial Sequence
  Exemplary nucleic acid sequence encoding a CTA epitope
gcggatagccgcccgccggatgaaattaaacagagc

SEQ ID NO: 10 - protein, 12 aa, Artificial Sequence
  Exemplary CTA epitope
ADSRPPDEIKQS

SEQ ID NO: 11 - DNA, 33 bp, Artificial Sequence
  Exemplary nucleic acid sequence encoding a FlaB epitope
agcaacggcagcaacagcagcagcgaacgccgc

SEQ ID NO: 12 - protein, 11 aa, Artificial Sequence
  Exemplary FlaB epitope
SNGSNSSSERR

SEQ ID NO: 13 - DNA, 30 bp, Artificial Sequence
  Exemplary nucleic acid sequence encoding a CTB epitope
agccagcatattgatagccagaaaaaagcg

SEQ ID NO: 14 - protein, 10 aa, Artificial Sequence
  Exemplary CTB epitope
SQHIDSQKKA

SEQ ID NO: 15 - DNA, 36 bp, Artificial Sequence
  Exemplary nucleic acid sequence encoding an LPS O-antigen mimic epitope
ctgccgagcgcgggccgcggcgtgtgctatgaagcg

SEQ ID NO: 16 - protein, 12 aa, Artificial Sequence
  Exemplary LPS O-antigen mimic epitope
LPSAGRGVCYEA

SEQ ID NO: 17 - DNA, 33 bp, Artificial Sequence
  Exemplary nucleic acid sequence encoding a sialidase epitope
atgcaggataacaccaacaacggcagcggcgtg

SEQ ID NO: 18 - protein, 11 aa, Artificial Sequence
  Exemplary sialidase epitope
MQDNTNNGSGV

SEQ ID NO: 19 - DNA, 36 bp, Artificial Sequence
  Exemplary nucleic acid sequence encoding a HlyA epitope
accggcggcgtggaagtgagcggcgatggcccgaaa

SEQ ID NO: 20 - protein, 12 aa, Artificial Sequence
  Exemplary HlyA epitope
TGGVEVSGDGPK

SEQ ID NO: 21 - DNA, 42 bp, Artificial Sequence
  Exemplary nucleic acid sequence encoding a FlaC/D epitope
ctgcagagccagagcgcgaacggcagcaacagcaaaagcgaa

SEQ ID NO: 22 - protein, 14 aa, Artificial Sequence
  Exemplary FlaC/D epitope
LQSQSANGSNSKSE

SEQ ID NO: 23 - DNA, 33 bp, Artificial Sequence
  Exemplary nucleic acid sequence encoding an LPS O-antigen mimic epitope
cagcatctgaacagcattctgctggtgaccaaa

SEQ ID NO: 24 - protein, 11 aa, Artificial Sequence
  Exemplary LPS O-anitgen mimic epitope
QHLNSILLVTK

SEQ ID NO: 25 - DNA, 480 bp, Artificial Sequence
  Exemplary nucleic acid sequence encoding a fusion protein with ETEC antigens
atggaaatggctagcgcagtagaggattttttcattgttccagtttctggagatcctgcaattgatcttttgcaagctgatggcaatgctctgccatcagctgtaaagttagcttattctcccgcatcaaaaactaatactttggtgggtgttttgactcttgtacatacaaacgatgcaactaaaaaaaatgtactagttaagcttgtaacaccacagcttacagatgttctgaatccaaccctgcaaattcctgtttctgtgcaggtaacggtctaccctgtttctacaacagccaaagaatttgaagctgctgctttgggatattctgcatccggtgtaaatggcttggtgtcaattgtgcttactgtaattagcgctgcacctaaaactgccggtaccgccccaactgcaggaaactattcaggagtagtatctcttgtaatgactttgggagcctgataacggccgcactcgagc

SEQ ID NO: 26 - protein, 153 aa, Artificial Sequence
  Exemplary fusion protein with ETEC antigens
MEMASAVEDFFIVPVSGDPAIDLLQADGNALPSAVKLAYSPASKTNTLVGVLTLVHTNDATKKNVLVKLVTPQLTDVLNPTLQIPVSVQVTVYPVSTTAKEFEAAALGYSASGVNGLVSIVLTVISAAPKTAGTAPTAGNYSGVVSLVMTLGA

SEQ ID NO: 27 - DNA, 1278 bp, Artificial Sequence
  Exemplary nucleic acid sequence encoding a fusion protein with ETEC toxoid antigen
atggaaatggctagcatgaatagtagcaattactgctgtgaattgtgttgtagccctgcttgtaccgggtgctatgggccggggcccaatggcgacaaattataccgtgctgactctagacccccagatgaaataaaacgttccggaggtcttatgcccagagggcataatgagtacttcgatagaggaactcaaatgaatattaatctttatgatcacgcgagaggaacacaaaccggctttgtcagatatgatgacggatatgtttccacttctcttagtttgagaagtgctcacttagcaggacagtctatattatcaggatattccacttactatatatatgttatagcgacagcaccaaatatgtttaatgttaatgatgtattaggcgtatacagccctcacccatatgaacaggaggtttctgcgttaggtggaataccatattctcagatatatggatggtatcgtgttaattttggtgtgattgatgaacgattacatcgtaacagggaatatagagaccggtattacagaaatctgaatatagctccggcagaggatggttacagattagcaggtttcccaccggatcaccaagcttggagagaagaaccctggattcatcatgcaccacaaggttgtggaaattcatcaggagggccggtcgacatgaatagtagcaattactgctgtgaattgtgttgtagccctgcttgtaccgggtgctatacaattacaggtgatacttgtaatgaggagacccagaatctgagcacaatatatgccaggaaatatcaatcaaaagttaagaggcagatattttcagactatcagtcagaggttgacatatataacagaattcggaatgaattagggccggggcccgctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagaccaaaattgataaattatgtgtatggaataataaaacccccaattcaattgcggcaatcagtgatccccgggtaccgagctcgatgaatagtagcaattactgctgtgaattgtgttgtagccctgcttgtaccgggtgctattaataa

SEQ ID NO: 28 - protein, 424 aa, Artificial Sequence
  Exemplary fusion protein with ETEC toxoid antigen
MEMASMNSSNYCCELCCSPACTGCYGPGPNGDKLYRADSRPPDEIKRSGGLMPRGHNEYFDRGTQMNINLYDHARGTQTGFVRYDDGYVSTSLSLRSAHLAGQSILSGYSTYYIYVIATAPNMFNVNDVLGVYSPHPYEQEVSALGGIPYSQIYGWYRVNFGVIDERLHRNREYRDRYYRNLNIAPAEDGYRLAGFPPDHQAWREEPWIHHAPQGCGNSSGGPVDMNSSNYCCELCCSPACTGCYTITGDTCNEETQNLSTIYARKYQSKVKRQIFSDYQSEVDIYNRIRNELGPGPAPQSITELCSEYRNTQIYTINDKILSYTESMAGKREMVIITFKSGATFQVEVPGSQHIDSQKKAIERMKDTLRITYLTETKIDKLCVWNNKTPNSIAAISDPRVPSSMNSSNYCCELCCSPACTGCY

SEQ ID NO: 29 - DNA, 1014 bp, Artificial Sequence
  Exemplary nucleic acid sequence encoding a fusion protein with shigella epitopes
atgggcaatataacaactctgactaatagtatttccacctcatcattcagcccaaacaataccaacggttcatcaaccgaaacagttaattctgatataaaaacaacgaccagttctcatcctgtaagttcccttactatgctcaacgacacccttcataatatcagaacaacaaatcaggcattaaagaaagatctctcacaaaaaacgttgactaaaacatcgctagaagaaatagcattacattcatctcagattagcatggatgtaaacaaatccgctcaactattggatattctttccaagaaagaatatccaattaataaagacgcaagagaattattacattcagcccaactagttggaaaaaataatgaagaatctatgatatctcacagagaactgtgggataaaattgcaaagtcaatcaataatattaatgaacagtatctgaaagtatatgaacatgccgttagttcatatactcaatctgctgaacagctatcaacccagcagaaaagtcttgccggctggatctctcccggaggtaacgacggaaactccgtgaaattacaagtcaagtctcttaaaagtgatagtgatggggggaatggaggtgataaagataaaccgctatatccagcaaataatactgttctgcctgactatcatggacaagactctgttcgtgtagaattaggtggaacagaacgtaaaccgaacgcttgtaaagacgagtatgttgtcaatataaacatgaccccaatagacaatatgcttccggagtatcggggagaggatggtgtcagagtgggtgaggttgtgctagataatgcaaaatatcaggcatggaatgccggagagtatacaaaatataatgatgacgataccaatcttcaaaccttagttcaaaaatacagtaatgccaatagtatttttgataatttagtaaaggttttgagtagtacaataagttcatgtacagatacagataaactttttctccatttctga

SEQ ID NO: 30 - protein, 337 aa, Artificial Sequence
  Exemplary fusion protein with shigella epitopes
MGNITTLTNSISTSSFSPNNTNGSSTETVNSDIKTTTSSHPVSSLTMLNDTLHNIRTTNQALKKDLSQKTLTKTSLEEIALHSSQISMDVNKSAQLLDILSKKEYPINKDARELLHSAQLVGKNNEESMISHRELWDKIAKSINNINEQYLKVYEHAVSSYTQSAEQLSTQQKSLAGWISPGGNDGNSVKLQVKSLKSDSDGGNGGDKDKPLYPANNTVLPDYHGQDSVRVELGGTERKPNACKDEYVVNINMTPIDNMLPEYRGEDGVRVGEVVLDNAKYQAWNAGEYTKYNDDDTNLQTLVQKYSNANSIFDNLVKVLSSTISSCTDTDKLFLHF

SEQ ID NO: 31 - protein, 9 aa, Artificial Sequence
  Exemplary LPS O-antigen mimic epitope
CFFPNLSYC

SEQ ID NO: 32 - protein, 12 aa, Artificial Sequence
  Exemplary LPS O-antigen mimic epitope
SMCMHGGAYCFP

SEQ ID NO: 33 - DNA, 34 bp, Artificial Sequence
  Exemplary primer sequence
cccatgggcatgacattactcgaagtaatcattg

SEQ ID NO: 34 - DNA, 34 bp, Artificial Sequence
  Exemplary primer sequence
tcacggccgttaactgttaccaaaagctactgtg

SEQ ID NO: 35 - DNA, 28 bp, Artificial Sequence
  Exemplary primer sequence
ccgccatggatgcaattattaaaacagc

SEQ ID NO: 36 - DNA, 32 bp, Artificial Sequence
  Exemplary primer sequence
tcacggccgttaactgttaccaaaagctactg

SEQ ID NO: 37 - DNA, 30 bp, Artificial Sequence
  Exemplary primer sequence
ctagctagcatggcaattaatgtaaacacg

SEQ ID NO: 38 - DNA, 34 bp, Artificial Sequence
  Exemplary primer sequence
tcacggccgttattatcccaataagctcagagct

SEQ ID NO: 39 - DNA, 30 bp, Artificial Sequence
  Exemplary primer sequence
ctagctagcatggcggtgaatgtaaacacc

SEQ ID NO: 40 - DNA, 30 bp, Artificial Sequence
  Exemplary primer sequence
tcacggccgctagcctagaagagccagtgc

SEQ ID NO: 41 - DNA, 29 bp, Artificial Sequence
  Exemplary primer sequence
ctagctagcatggcagtgaatgtaaatac

SEQ ID NO: 42 - DNA, 32 bp, Artificial Sequence
  Exemplary primer sequence
tcacggccgttattaacccaacaggctgagtg

SEQ ID NO: 43 - DNA, 33 bp, Artificial Sequence
  Exemplary primer sequence
cggaattccgatggcaacgggtgacactgagtt

SEQ ID NO: 44 - DNA, 33 bp, Artificial Sequence
  Exemplary primer sequence
tcacggccgttattaggtatcccaagttatacc

SEQ ID NO: 45 - DNA, 26 bp, Artificial Sequence
  Exemplary primer sequence
ccgccatggatgccaaaactcaatcg

SEQ ID NO: 46 - DNA, 27 bp, Artificial Sequence
  Exemplary primer sequence
tcacggccggttatcgacaggaaaggt